(19) United States
(12) Patent Application Publication
(10) Pub. No.: US 2026/0274981 A1
(43) Pub. Date: Sep. 17, 2026

(54) Blood-Brain Barrier Crossing Antibodies

(71) Applicants: VIB VZW, Gent (BE); Katholieke Universiteit Leuven, Leuven (BE)

(72) Inventors: Bart De Strooper, Leuven (BE); Bruno Dombrecht, Gent (BE); Maarten DeWilde, Leuven (BE); Tom Jaspers, Leuven (BE); Laura Rué, Leuven (BE)

(21) Appl. No.: 19/471,817

(22) PCT Filed: Apr. 2, 2024
(86) PCT No.: PCT/EP2024/058918

§ 371 Date: Oct. 2, 2025

(30) Foreign Application Priority Data
Apr. 3, 2023 (EP) 23166341.0

(51) Int. Cl. C07K 16/28; A61K 47/68; C07K 16/40

(52) U.S. Cl. CPC C07K 16/2881; A61K 47/6811; A61K 47/6849; C07K 16/40; C07K 2317/22; C07K 2317/569

(57) ABSTRACT

The present invention relates to antibodies or antibody fragments binding to the human and non-humane primate transferrin receptor. The antibodies herein described can be used as agents to deliver pharmaceutical compounds in or across cells in the process of receptor mediated endocytosis and/or transcytosis. As the transferrin receptor is also present at the blood brain barrier endothelial cells, one aspect of the invention provides means and methods to increase the delivery of pharmaceutical compounds to the central nervous system.

### CROSS REFERENCE TO RELATED APPLICATIONS

This application is a national phase entry under 35 U.S.C. § 371 of International Patent Application PCT/EP2024/058918, filed Apr. 2, 2024, designating the United States of America and published in English as International Patent Publication WO2024/208816 on Oct. 10, 2024, which claims the benefit under Article 8 of the Patent Cooperation Treaty to European Patent Application Serial No. 23166341.0, filed Apr. 3, 2023, the entireties of which are hereby incorporated by reference.

### INCORPORATION BY REFERENCE

The ST.26 XML Sequence listing named “10488-11096-US-SequenceListingST26”, created on Mar. 28, 2024, and having a size of 69,632 bytes, is hereby incorporated herein by this reference in its entirety.

### FIELD OF THE INVENTION

The present invention relates to antibodies or antibody fragments binding the human and non-human primate transferrin receptors (TfR). The antibodies and the methods herein described can be used to increase the delivery of pharmaceutical compounds to the central nervous system in the process of receptor mediated endocytosis and/or transcytosis.

### BACKGROUND

The central nervous system (CNS) is isolated from the rest of the organism by a very specialized organ, the blood-brain barrier (BBB), which protects it from harmful circulating substances in the peripheral blood flow while still allowing selective influx of required elements such as nutrients. Hence the BBB represents a bottleneck for the treatment of neurological diseases, as most of the biologicals are not able to reach their brain targets (Freskgard & Ulrich 2017 Neuropharmacology 120, 28-55). In the best scenario, they reach their brain targets in very small quantities, and therefore high doses of biologicals need to be administered, resulting in potential side effects and high treatment costs (Freskgard & Ulrich 2017 Neuropharmacology 120, 28-55; St-Amour et al 2013 J Cereb Blood Flow Metab 33, 1983-1992; Poduslo et al 1994 PNAS 91, 5705-5709). The blood-brain barrier is composed of an endothelial layer which is surrounded by pericytes and astrocyte endfeet. In contrast to other endothelia in the organism, the endothelium in the BBB expresses tight junctions that limit paracellular diffusion of substances. Instead, most of the required substances in the brain that come from the periphery follow an active route of entry by specific channels and transporters. Receptor-mediated transcytosis (RMT) is one such physiological mechanism in which nutrients are recognized by specific receptors expressed on the surface of the endothelial cells, internalized in intracellular vesicles and finally released in the brain parenchyma. Targeting such RMT receptors with antibodies has been proven to be a valid strategy to increase the brain permeability of biologicals (Pardridge 1986 Endocrine Reviews 7, 314-330), and transferrin receptor (TfR), is one of the most exploited RMT mechanisms for brain drug delivery (Sehlin et al 2020 FASEB J 34, 13272-13283; Su et al 2022 PLoS One 17; Sonoda et al 2018 Molecular Therapy 26, 1366-1374). Recently one anti-TfR-idursulfase conjugate drug (Izcargo®) was approved in Japan for the treatment of Hunter syndrome (Giugliani et al 2021 Molecular Therapy 29, 2378-2386).

We previously identified TfR nanobodies that successfully delivered biologicals over the BBB. We obtained a set of mouse TfR binders and also a set of human TfR binders (Wouters et al 2020 Fluids Barriers CNS 17, 62; Wouters et al 2020 Fluids Barriers CNS 19, 79). Unfortunately, our human TfR nanobodies did not bind non-human primate (NHP) TfR, despite the high sequence homology between both proteins. Lack of binding to NHP TfR represents an obstacle to determine preclinical efficacy and safety of potential therapeutic conjugates. In current application, the identification of two human/cynomolgus TfR binding nanobodies is discloses as well as the validation in vivo of their potential to shuttle therapeutics into the brain. More particularly single domain antibodies, more particularly VHHs are disclosed that bind the human and NHP transferrin receptor (TfR). The herein described antibodies can deliver compounds including therapeutic and/or diagnostic antibodies and small molecules across the BBB after a single systemic administration in mice. The VHH sequences described herein were compared to VHH sequences binding to TfR disclosed in patent application WO2020144233 (Vect-Horus). From a therapeutic biologics developability perspective, the Vect-Horus VHHs display a number of severe liabilities and the instant VHHs disclosed herein have an improved chemical stability profile, have a higher thermal stability and have a lower oligomerization and lower aggregation propensities.

### BRIEF DESCRIPTION OF THE FIGURES

FIGS. 1A-1F summarizes the identification of human/cynomolgus TfR binders. FIG. 1A shows the immunization, selection and screening strategy followed to select human and cynomolgus TfR nanobody binders. FIGS. 1B-1E shows the binding of the VHHs to CHO cells overexpressing hTfR (FIG. 1B), cynomolgus TfR (FIG. 1C), mouse TfR (FIG. 1D) and GFP (FIG. 1E). FIG. 1F summarizes the results of the kinetic analysis of the VHHs binding to cynomolgus and human TfR recombinant material assessed with SPR.

FIGS. 2A-2F shows the shuttling of the anti-TfR/anti-BACE1 composition across the BBB. FIG. 2A is an illustration of the bispecific antibody design. FIGS. 2B and 2C shows the bispecific antibody binding to human TfR (FIG. 2B) or GFP overexpressing cells (FIG. 2C). FIGS. 1D-1F summarizes the BLI kinetic analysis of the antibodies binding to BACE1.

FIGS. 3A and 3B shows the Aβ40 levels in plasma (FIG. 3A) and brains (FIG. 3B) of human TfR knock-in mice as readout of BACE1 inhibition upon peripheral administration of the VHHs of the application couple to anti-BACE1.

FIG. 4 shows the ASEC profile of exemplary humanized variants disclosed in WO2020144233 (Vect-Horus) compared with BBB00515 and BBB00533.

FIG. 5 shows the SYPRO orange fluorescence spectrum of exemplary humanized variants disclosed in WO2020144233 (Vect-Horus) compared with BBB00515 and BBB00533.

FIG. 6 depicts a sequence alignment of BBB00515 and its humanized variants. The CDR1, 2 and 3 regions are shaded in grey.

FIG. 7 depicts a sequence alignment of BBB00533 and its humanized variants. The CDR1, 2 and 3 regions are shaded in grey.

FIG. 8 depicts the binding characteristics and biophysical parameters of the humanized variants of BBB00533.

FIG. 9 depicts the binding characteristics and biophysical parameters of the humanized variants of BBB00515.

FIG. 10 shows the pH dependent binding with human TfR for the histidine mutants of BBB00515.

FIG. 11 shows the pH dependent binding with human TfR for the histidine mutants of BBB00533.

FIGS. 12A and 12B depicts the details and alignment of the histidine mutants for BBB00515 and BBB00533.

### DETAILED DESCRIPTION

### Definitions

In order that the present description can be more readily understood, certain terms are first defined. Additional definitions are set forth throughout the detailed description. The present invention is described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. It is to be noted that the term “a” or “an” entity refers to one or more of that entity; for example, “a nucleotide sequence”, is understood to represent one or more nucleotide sequences. As such, the terms “a” (or “an”), “one or more” and “at least one” can be used interchangeably herein. Furthermore, “and/or” where used herein is to be taken as specific disclosure of each of the two specified features or components with or without the other. Thus, the term “and/or” as used in a phrase such as “A and/or B” herein is intended to include “A and B”, “A or B”, “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone). Where an indefinite or definite article is used when referring to a singular noun e.g. “a” or “an”, “the”, this includes a plural of that noun unless something else is specifically stated. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

It is understood that wherever aspects or embodiments are described herein with the language “comprising”, otherwise analogous aspects or embodiments described in terms of “consisting of” and/or “consisting essentially of” are also provided. Where the term “comprising” is used in the present description and claims, it does not exclude other elements or steps. Unless specifically defined herein, all terms used herein have the same meaning as they would to one skilled in the art of the present invention.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure. Practitioners are particularly directed to Sambrook et al., Molecular Cloning: A Laboratory Manual, 4th ed., Cold Spring Harbor Press, Plainsview, New York (2012); and Ausubel et al., current Protocols in Molecular Biology (Supplement 100), John Wiley & Sons, New York (2012), for definitions and terms of the art. The definitions provided herein should not be construed to have a scope less than understood by a person of ordinary skill in the art.

Units, prefixes, and symbols are denoted in their Systeme International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance. For example a dissociation constant koff of about 1.50×10⁻²/s implies that the koff is within the range between 1.45×10⁻² to 1.55×10⁻²/s.

The present application relates to antibodies binding the human and NHP transferrin receptor.

The term “antibody” as used herein, refers to an immunoglobulin (Ig) molecule or a molecule comprising an immunoglobulin (Ig) domain, which specifically binds with an antigen. “Antibodies” can be intact immunoglobulins derived from natural sources or from recombinant sources and can be immunoreactive portions of intact immunoglobulins. Antibodies are typically tetramers of immunoglobulin molecules. The term “immunoglobulin (Ig) domain” as used herein refers to a globular region of an antibody chain, or to a polypeptide that essentially consists of such a globular region. Immunoglobulin domains are characterized in that they retain the immunoglobulin fold (Ig fold as named herein) characteristic of antibody molecules, which consists of a two-layer sandwich of about seven to nine antiparallel β-strands arranged in two β-sheets, optionally stabilized by a conserved disulphide bond. The term “immunoglobulin (Ig) domain”, includes “immunoglobulin constant domain”, and “immunoglobulin variable domain” (abbreviated as “IVD”), wherein the latter means an immunoglobulin domain essentially consisting of four “framework regions” which are referred to in the art and herein below as “framework region 1” or “FR1”; as “framework region 2” or “FR2”; as “framework region 3” or “FR3”; and as “framework region 4” or “FR4”, respectively; which framework regions are interrupted by three “complementarity determining regions” or “CDRs”, which are referred to in the art and herein below as “complementarity determining region 1” or “CDR1”; as “complementarity determining region 2” or “CDR2”; and as “complementarity determining region 3” or “CDR3”, respectively. Thus, the general structure or sequence of an immunoglobulin variable domain can be indicated as follows: FR1-CDR1-FR2-CDR2-FR3-CDR3-FR4. It is the immunoglobulin variable domain(s) (IVDs) that confer specificity to an antibody for the antigen by carrying the antigen-binding site.

An “immunoglobulin domain” of this application also includes “immunoglobulin single variable domains” (abbreviated as “ISVD”), equivalent to the term “single variable domains”, and defines molecules wherein the antigen binding site is present on, and formed by, a single immunoglobulin domain. This sets immunoglobulin single variable domains apart from “conventional” immunoglobulins or their fragments, wherein two immunoglobulin domains, in particular two variable domains, interact to form an antigen binding site. Typically, in conventional immunoglobulins, a heavy chain variable domain (VH) and a light chain variable domain (VL) interact to form an antigen binding site. In this case, the complementarity determining regions (CDRs) of both VH and VL will contribute to the antigen binding site, i.e. a total of 6 CDRs will be involved in antigen binding site formation. In view of the above definition, the antigen-binding domain of a conventional 4-chain antibody (such as an IgG, IgM, IgA, IgD or IgE molecule; known in the art) or of a Fab fragment, a F(ab′)2 fragment, an Fv fragment such as a disulphide linked Fv or a scFv fragment, or a diabody (all known in the art) derived from such conventional 4-chain antibody, would normally not be regarded as an immunoglobulin single variable domain, as, in these cases, binding to the respective epitope of an antigen would normally not occur by one (single) immunoglobulin domain but by a pair of (associated) immunoglobulin domains such as light and heavy chain variable domains, i.e., by a VH-VL pair of immunoglobulin domains, which jointly bind to an epitope of the respective antigen. In contrast, immunoglobulin single variable domains are capable of specifically binding to an epitope of the antigen without pairing with an additional immunoglobulin variable domain. The binding site of an immunoglobulin single variable domain is formed by a single VH/VHH or VL domain. Hence, the antigen binding site of an immunoglobulin single variable domain is formed by no more than three CDRs. As such, the single variable domain may be a light chain variable domain sequence (e.g., a VL-sequence) or a suitable fragment thereof; or a heavy chain variable domain sequence (e.g., a VH-sequence or VHH sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit). In one embodiment of the invention, the immunoglobulin single variable domains are heavy chain variable domain sequences (e.g., a VH-sequence); more specifically, the immunoglobulin single variable domains can be heavy chain variable domain sequences that are derived from a conventional four-chain antibody or heavy chain variable domain sequences that are derived from a heavy chain antibody. For example, the immunoglobulin single variable domain may be a (single) domain antibody (or an amino acid sequence that is suitable for use as a (single) domain antibody), a “dAb” or dAb (or an amino acid sequence that is suitable for use as a dAb) or a Nanobody (as defined herein, and including but not limited to a VHH); other single variable domains, or any suitable fragment of any one thereof. In particular, the immunoglobulin single variable domain may be a Nanobody (as defined herein) or a suitable fragment thereof. Note: Nanobody®, Nanobodies® and Nanoclone® are registered trademarks of Ablynx N.V. For a general description of Nanobodies, reference is made to the further description below, as well as to the prior art cited herein, such as e.g. described in WO2008/020079.

Immunoglobulin domains herein also include “VHH domains”, also known as VHHs, VHH domains, VHH antibody fragments, and VHH antibodies, have originally been described as the antigen-binding immunoglobulin (Ig) (variable) domain of “heavy chain antibodies” (i.e., of “antibodies devoid of light chains”; Hamers-Casterman et al (1993) Nature 363: 446-448). The term “VHH domain” has been chosen to distinguish these variable domains from the heavy chain variable domains that are present in conventional 4-chain antibodies (which are referred to herein as “VH domains”) and from the light chain variable domains that are present in conventional 4-chain antibodies (which are referred to herein as “VL domains”). For a further description of VHHs and Nanobody, reference is made to the review article by Muyldermans (Reviews in Molecular Biotechnology 74: 277-302, 2001), as well as to the following patent applications, which are mentioned as general background art: WO 94/04678, WO 95/04079 and WO 96/34103 of the Vrije Universiteit Brussel; WO 94/25591, WO 99/37681, WO 00/40968, WO 00/43507, WO 00/65057, WO 01/40310, WO 01/44301, EP 1134231 and WO 02/48193 of Unilever; WO 97/49805, WO 01/21817, WO 03/035694, WO 03/054016 and WO 03/055527 of the Vlaams Instituut voor Biotechnologie (VIB); WO 03/050531 of Algonomics N.V. and Ablynx N.V.; WO 01/90190 by the National Research Council of Canada; WO 03/025020 (=EP 1433793) by the Institute of Antibodies; as well as WO 04/041867, WO 04/041862, WO 04/041865, WO 04/041863, WO 04/062551, WO 05/044858, WO 06/40153, WO 06/079372, WO 06/122786, WO 06/122787 and WO 06/122825, by Ablynx N.V. and the further published patent applications by Ablynx N.V. As described in these references, Nanobody (in particular VHH sequences and partially humanized Nanobody) can in particular be characterized by the presence of one or more “Hallmark residues” in one or more of the framework sequences. A further description of the Nanobody, including humanization and/or camelization of Nanobody, as well as other modifications, parts or fragments, derivatives or “Nanobody fusions”, multivalent constructs (including some non-limiting examples of linker sequences) and different modifications to increase the half-life of the Nanobody and their preparations can be found e.g. in WO 08/101985 and WO 08/142164.

“Domain antibodies”, also known as “Dabs”, “Domain Antibodies”, and “dAbs” (the terms “Domain Antibodies” and “dAbs” being used as trademarks by the GlaxoSmithKline group of companies) have been described in e.g., EP 0368684, Ward et al. (Nature 341: 544-546, 1989), Holt et al. (Tends in Biotechnology 21: 484-490, 2003) and WO 03/002609 as well as for example WO 04/068820, WO 06/030220, WO 06/003388 and other published patent applications of Domantis Ltd. Domain antibodies essentially correspond to the VH or VL domains of non-camelid mammalians, in particular human 4-chain antibodies. In order to bind an epitope as a single antigen binding domain, i.e., without being paired with a VL or VH domain, respectively, specific selection for such antigen binding properties is required, e.g. by using libraries of human single VH or VL domain sequences. Domain antibodies have, like VHHs, a molecular weight of approximately 13 to approximately 16 kDa and, if derived from fully human sequences, do not require humanization for e.g. therapeutical use in humans. It should also be noted that single variable domains can be derived from certain species of shark (for example, the so-called “IgNAR domains”, see for example WO 05/18629).

Immunoglobulin single variable domains such as Domain antibodies and Nanobody (including VHH domains and humanized VHH domains), represent in vivo matured macromolecules upon their production, but can be further subjected to affinity maturation by introducing one or more alterations in the amino acid sequence of one or more CDRs, which alterations result in an improved affinity of the resulting immunoglobulin single variable domain for its respective antigen, as compared to the respective parent molecule. Affinity-matured immunoglobulin single variable domain molecules of the invention may be prepared by methods known in the art, for example, as described by Marks et al. (Biotechnology 10:779-783, 1992), Barbas et al. (Proc. Nat. Acad. Sci, USA 91: 3809-3813, 1994), Shier et al. (Gene 169: 147-155, 1995), Yelton et al. (Immunol. 155: 1994-2004, 1995), Jackson et al. (J. Immunol. 154: 3310-9, 1995), Hawkins et al. (J. Mol. Biol. 226: 889 896, 1992), Johnson and Hawkins (Affinity maturation of antibodies using phage display, Oxford University Press, 1996). The process of designing/selecting and/or preparing a polypeptide, starting from an immunoglobulin single variable domain such as a Domain antibody or a Nanobody, is also referred to herein as “formatting” said immunoglobulin single variable domain; and an immunoglobulin single variable domain that is made part of a polypeptide is said to be “formatted” or to be “in the format of” said polypeptide. Examples of ways in which an immunoglobulin single variable domain can be formatted and examples of such formats for instance to avoid glycosylation will be clear to the skilled person based on the disclosure herein.

Immunoglobulin single variable domains such as Domain antibodies and Nanobody® (including VHH domains) can be subjected to humanization, i.e. increase the degree of sequence identity with the closest human germline sequence. In particular, humanized immunoglobulin single variable domains, such as Nanobody® (including VHH domains) may be immunoglobulin single variable domains in which at least one amino acid residue is present (and in particular, at least one framework residue) that is and/or that corresponds to a humanizing substitution (as defined further herein). Potentially useful humanizing substitutions can be ascertained by comparing the sequence of the framework regions of a naturally occurring VHH sequence with the corresponding framework sequence of one or more closely related human VH sequences, after which one or more of the potentially useful humanizing substitutions (or combinations thereof) thus determined can be introduced into said VHH sequence (in any manner known per se, as further described herein) and the resulting humanized VHH sequences can be tested for affinity for the target, for stability, for ease and level of expression, and/or for other desired properties. In this way, by means of a limited degree of trial and error, other suitable humanizing substitutions (or suitable combinations thereof) can be determined by the skilled person. Also, based on what is described before, (the framework regions of) an immunoglobulin single variable domain, such as a Nanobody® (including VHH domains) may be partially humanized or fully humanized.

Humanized immunoglobulin single variable domains, in particular Nanobody®, may have several advantages, such as a reduced immunogenicity, compared to the corresponding naturally occurring VHH domains. By humanized is meant mutated so that immunogenicity upon administration in human patients is minor or non-existent. The humanizing substitutions should be chosen such that the resulting humanized amino acid sequence and/or VHH still retains the favourable properties of the VHH, such as the antigen-binding capacity. Based on the description provided herein, the skilled person will be able to select humanizing substitutions or suitable combinations of humanizing substitutions which optimize or achieve a desired or suitable balance between the favourable properties provided by the humanizing substitutions on the one hand and the favourable properties of naturally occurring VHH domains on the other hand. Such methods are known by the skilled addressee. A human consensus sequence can be used as target sequence for humanization, but also other means are known in the art. One alternative includes a method wherein the skilled person aligns a number of human germline alleles, such as for instance but not limited to the alignment of IGHV3 alleles, to use said alignment for identification of residues suitable for humanization in the target sequence. Also a subset of human germline alleles most homologous to the target sequence may be aligned as starting point to identify suitable humanisation residues. Alternatively, the VHH is analyzed to identify its closest homologue in the human alleles, and used for humanisation construct design. A humanisation technique applied to Camelidae VHHs may also be performed by a method comprising the replacement of specific amino acids, either alone or in combination. Said replacements may be selected based on what is known from literature, are from known humanization efforts, as well as from human consensus sequences compared to the natural VHH sequences, or the human alleles most similar to the VHH sequence of interest. As can be seen from the data on the VHH entropy and VHH variability given in Tables A-5-A-8 of WO 08/020079, some amino acid residues in the framework regions are more conserved between human and Camelidae than others. Generally, although the invention in its broadest sense is not limited thereto, any substitutions, deletions or insertions are preferably made at positions that are less conserved. Also, generally, amino acid substitutions are preferred over amino acid deletions or insertions. For instance, a human-like class of Camelidae single domain antibodies contain the hydrophobic FR2 residues typically found in conventional antibodies of human origin or from other species, but compensating this loss in hydrophilicity by other substitutions at position 103 that substitutes the conserved tryptophan residue present in VH from double-chain antibodies. As such, peptides belonging to these two classes show a high amino acid sequence homology to human VH framework regions and said peptides might be administered to a human directly without expectation of an unwanted immune response therefrom, and without the burden of further humanisation. Indeed, some Camelidae VHH sequences display a high sequence homology to human VH framework regions and therefore said VHH might be administered to patients directly without expectation of an immune response therefrom, and without the additional burden of humanization.

Suitable mutations, in particular substitutions, can be introduced during humanization to generate a polypeptide with reduced binding to pre-existing antibodies (reference is made for example to WO 2012/175741 and WO2015/173325), for example at at least one of the positions: 11, 13, 14, 15, 40, 41, 42, 82, 82a, 82b, 83, 84, 85, 87, 88, 89, 103, or 108. The amino acid sequences and/or VHH of the invention may be suitably humanized at any framework residue(s), such as at one or more Hallmark residues (as defined below) or at one or more other framework residues (i.e. non-Hallmark residues) or any suitable combination thereof. Depending on the host organism used to express the amino acid sequence, VHH or polypeptide of the invention, such deletions and/or substitutions may also be designed in such a way that one or more sites for posttranslational modification (such as one or more glycosylation sites) are removed, as will be within the ability of the person skilled in the art. Alternatively, substitutions or insertions may be designed so as to introduce one or more sites for attachment of functional groups (as described herein), for example to allow site-specific pegylation.

In some cases, at least one of the typical Camelidae hallmark residues with hydrophilic characteristics at position 37, 44, 45 and/or 47 is replaced (see WO2008/020079 Table A-03). Another example of humanization includes substitution of residues in FR 1, such as position 1, 5, 11, 14, 16, and/or 28; in FR3, such as positions 73, 74, 75, 76, 78, 79, 82b, 83, 84, 93 and/or 94; and in FR4, such as position 103, 104, 108 and/or 111 (see WO2008/020079 Tables A-05-A08; all numbering according to the Kabat).

An “epitope”, as used herein, refers to an antigenic determinant of a polypeptide, constituting a binding site or binding pocket on a target molecule (e.g. a protein to which an immunoglobulin or part thereof, antibody, VHH or ISVD is binding). “Binding” means any interaction, be it direct or indirect. A direct interaction implies a contact (e.g. physical or chemical) between two binding partners. An indirect interaction means any interaction whereby the interaction partners interact in a complex of more than two molecules. An interaction can be completely indirect (e.g. two molecules are part of the same complex with the help of one or more bridging molecules but don't bind in the absence of the bridging molecule(s)). An interaction may be partly direct or partly indirect: there is still a direct contact between two interaction partners, but such contact is e.g. not stable, and is stabilized by the interaction with one or more additional molecules. The term “binding pocket” or “binding site” refers to a region of a molecule or molecular complex, that, as a result of its shape and charge, associates with another chemical entity, compound, protein, peptide, antibody, single domain antibody or ISVD or VHH.

An epitope could comprise 1, 2 or 3 amino acids in a spatial conformation, which is unique to the epitope. Generally, an epitope consists of at least 4, 5, 6, 7 such amino acids, and more usually, consists of at least 8, 9, 10 such amino acids. Methods of determining the spatial conformation of amino acids are known in the art, and include, for example, X-ray crystallography and multi-dimensional nuclear magnetic resonance. A “conformational epitope”, as used herein, refers to an epitope comprising amino acids in a spatial conformation that is unique to a folded 3-dimensional conformation of a polypeptide. Generally, a conformational epitope consists of amino acids that are discontinuous in the linear sequence but that come together in the folded structure of the protein. However, a conformational epitope may also consist of a linear sequence of amino acids that adopts a conformation that is unique to a folded 3-dimensional conformation of the polypeptide (and not present in a denatured state). In protein complexes, conformational epitopes consist of amino acids that are discontinuous in the linear sequences of one or more polypeptides that come together upon folding of the different folded polypeptides and their association in a unique quaternary structure. Similarly, conformational epitopes may here also consist of a linear sequence of amino acids of one or more polypeptides that come together and adopt a conformation that is unique to the quaternary structure. The term “conformation” or “conformational state” of a protein refers generally to the range of structures that a protein may adopt at any instant in time. One of skill in the art will recognize that determinants of conformation or conformational state include a protein's primary structure as reflected in a protein's amino acid sequence (including modified amino acids) and the environment surrounding the protein. The conformation or conformational state of a protein also relates to structural features such as protein secondary structures (e.g., α-helix, β-sheet, among others), tertiary structure (e.g., the 3-dimensional folding of a polypeptide chain), and quaternary structure (e.g., interactions of a polypeptide chain with other protein subunits). Posttranslational and other modifications to a polypeptide chain such as ligand binding, phosphorylation, sulfation, glycosylation, or attachments of hydrophobic groups, among others, can influence the conformation of a protein. Furthermore, environmental factors, such as pH, salt concentration, ionic strength, and osmolality of the surrounding solution, and interaction with other proteins and co-factors, among others, can affect protein conformation. The conformational state of a protein may be determined by either functional assay for activity or binding to another molecule or by means of physical methods such as X-ray crystallography, NMR, or spin labelling, among other methods. For a general discussion of protein conformation and conformational states, one is referred to Cantor and Schimmel, Biophysical Chemistry, Part I: The Conformation of Biological. Macromolecules, W.H. Freeman and Company, 1980, and Creighton, Proteins: Structures and Molecular Properties, W.H. Freeman and Company, 1993.

A “paratope” as used herein refers to the antigen-binding site and is the part of an antibody which recognizes and binds to an antigen. The paratope of a TfR binding agent thus consists of the amino acid residues of the binding agent that binds the epitope of the TfR protein.

The term “affinity”, as used herein, generally refers to the degree to which an antibody or other binding protein (as defined further herein) binds to a target protein so as to shift the equilibrium of target protein and binding protein toward the presence of a complex formed by their binding. Thus, for example, where an antibody and an antigen are combined in relatively equal concentration, an antibody of high affinity will bind to the antigen so as to shift the equilibrium toward high concentration of the resulting complex. The equilibrium dissociation constant KD (or K_{D}) is commonly used to describe the affinity between a ligand and a target protein, or an antibody and its antigen. KD is the calculated ratio of k_{off}/kₒₙ, between the antibody and its antigen and thus measures the propensity of a complex to fall apart into its component molecules. The association constant (kₒₙ or kon) is used to characterize how quickly the antibody binds to its target. The dissociation constant (k_{off} or koff, also referred to as kdis, Kdis, Kd or kd) is used to measure how quickly an antibody dissociates from its target and is expressed as number of units that dissociated from a target per second. Hence, the lower koff is, the higher the affinity towards the target. koff and thus also KD is inversely related to affinity. A high affinity interaction is characterized by a low KD, a fast recognizing (high kon) and a strong stability of formed complexes (low koff). It will be appreciated that within the scope of the present application, the term “affinity” is used in the context of the antibody or antibody fragment that binds an epitope of the transferrin receptor TfR, more particularly the antibody or antibody fragment is “functional” in binding its target via the CDR regions of its immunoglobulin (Ig) domain.

“Amino acids” as used herein refer to the structural units (monomers) that make up proteins. They join together to form short polymer chains called peptides or longer chains called either polypeptides or proteins. These chains are linear and unbranched, with each amino acid residue within the chain attached to two neighbouring amino acids. Twenty amino acids encoded by the universal genetic code are naturally incorporated into polypeptides and are called proteinogenic or natural amino acids. Natural amino acids or naturally occurring amino acids are glycine (Gly or G), Alanine (Ala or A), Valine (Val or V), Leucine (Leu or L), Isoleucine (Ile or I), Methionine (Met or M), Proline (Pro or P), Phenylalanine (Phe or F), Tryptophan (Trp or W), Serine (Ser or S), Threonine (Thr or T), Asparagine (Asn or N), Glutamine (Gln or Q), Tyrosine (Tyr or Y), Cysteine (Cys or C), Lysine (Lys or K), Arginine (Arg or R), Histidine (His or H), Aspartic Acid (Asp or D) and Glutamic Acid (Glu or E).

As used herein, the terms “nucleic acid”, “nucleic acid sequence” or “nucleic acid molecule” are used interchangeably and refer to a polymeric form of nucleotides of any length, either deoxyribonucleotides or ribonucleotides, or analogs thereof. Nucleic acids may have any three-dimensional structure, and may perform any function, known or unknown. Non-limiting examples of nucleic acids include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular. The nucleic acid may comprise a promoter, an intron, an enhancer region, a polyadenylation site, a translation initiation site, 5′ or 3′ untranslated regions, a reporter gene, a selectable marker or the like. The nucleic acid may comprise single stranded or double stranded DNA or RNA. The nucleic acid may comprise modified bases or a modified backbone. A nucleic acid that is up to about 100 nucleotides in length, is often also referred to as an oligonucleotide. “Nucleotides” as used herein refer to the building blocks of oligonucleotides and polynucleotides, and for the purposes of the present invention include both naturally occurring and non-naturally occurring nucleotides. In nature, nucleotides, such as DNA and RNA nucleotides comprise a ribose sugar moiety, a nucleobase moiety and one or more phosphate groups (which are absent in nucleosides). A nucleotide without a phosphate group is called a “nucleoside” and is thus a compound comprising a nucleobase moiety and a sugar moiety. As used herein, “nucleobase” means a group of atoms that can be linked to a sugar moiety to create a nucleoside that is capable of incorporation into an oligonucleotide, and wherein the group of atoms is capable of bonding with a complementary naturally occurring nucleobase of another oligonucleotide or nucleic acid. Naturally occurring nucleobases of RNA or DNA comprise the purine bases adenine (A) and guanine (G), and the pyrimidine bases thymine (T), cytosine (C) and uracil (U).

“Nucleotide sequence”, “DNA sequence” or “nucleic acid molecule(s)” as used herein refers to a polymeric form of nucleotides of any length, either ribonucleotides or deoxyribonucleotides. This term refers only to the primary structure of the molecule. Thus, this term includes double- and single-stranded DNA, the (reverse) complement DNA, and RNA. It also includes known types of modifications, for example, methylation, “caps” substitution of one or more of the naturally occurring nucleotides with an analogue. By “nucleic acid construct” it is meant a nucleic acid sequence that has been constructed to comprise one or more functional units not found together in nature. Examples include circular, linear, double-stranded, extrachromosomal DNA molecules (plasmids), cosmids (plasmids containing COS sequences from lambda phage), viral genomes comprising non-native nucleic acid sequences, and the like. “Coding sequence” is a nucleotide sequence, which is transcribed into mRNA and/or translated into a polypeptide when placed under the control of appropriate regulatory sequences. The boundaries of the coding sequence are determined by a translation start codon at the 5′-terminus and a translation stop codon at the 3′-terminus. A coding sequence can include, but is not limited to mRNA, cDNA, recombinant nucleotide sequences or genomic DNA, while introns may be present as well under certain circumstances.

An “expression cassette” as used herein comprises any nucleic acid construct capable of directing the expression of a gene/coding sequence of interest, which is operably linked to a promoter of the expression cassette. Expression cassettes are generally DNA constructs preferably including (5′ to 3′ in the direction of transcription): a promoter region, a polynucleotide sequence, homologue, variant or fragment thereof operably linked with the transcription initiation region, and a termination sequence including a stop signal for RNA polymerase and a polyadenylation signal. It is understood that all of these regions should be capable of operating in biological cells, such as prokaryotic or eukaryotic cells, to be transformed. The promoter region comprising the transcription initiation region, which preferably includes the RNA polymerase binding site, and the polyadenylation signal may be native to the biological cell to be transformed or may be derived from an alternative source, where the region is functional in the biological cell. Such cassettes can be constructed into a “vector”. The term “vector” or alternatively “vector construct”, “expression vector” or “gene transfer vector” is intended to refer to a nucleic acid molecule capable of transporting another nucleic acid molecule to which it has been linked, and includes any vector known to the skilled person, including any suitable type, but not limited to, for instance, plasmid vectors, cosmid vectors, phage vectors, such as lambda phage, viral vectors, such as adenoviral, AAV or baculoviral vectors, or artificial chromosome vectors such as bacterial artificial chromosomes (BAC), yeast artificial chromosomes (YAC), or P1 artificial chromosomes (PAC). Expression vectors comprise plasmids as well as viral vectors and generally contain a desired coding sequence and appropriate DNA sequences necessary for the expression of the operably linked coding sequence in a particular host organism (e.g., bacteria, yeast, plant, insect, or mammal) or in in vitro expression systems. Cloning vectors are generally used to engineer and amplify a certain desired DNA fragment and may lack functional sequences needed for expression of the desired DNA fragments. The construction of expression vectors for use in transfecting cells is also well known in the art, and thus can be accomplished via standard techniques (see, for example, Sambrook, Fritsch, and Maniatis, in: Molecular Cloning, A Laboratory Manual, Cold Spring Harbor Laboratory Press, 1989; Gene Transfer and Expression Protocols, pp. 109-128, ed. E. J. Murray, The Humana Press Inc., Clif ton, N.J.), and the Ambion 1998 Catalog (Ambion, Austin, Tex.).

The terms “identical” or percent “identity” in the context of two or more nucleic acid or amino acid sequences refer to two or more sequences that are the same or have a specified percentage of nucleotides or amino acid residues respectively that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of nucleotide or amino acid sequences.

The term “percent sequence identity” or “% sequence identity” or “percent identity” or “% identity” between two polynucleotide or polypeptide sequences refers to the number of identical matched positions shared by the sequences over a comparison window, taking into account additions or deletions (i.e. gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence. One such non-limiting example of a sequence alignment algorithm is the algorithm described in Karlin et al., 1990, Proc. Natl. Acad. Sci., 87:2264-2268, as modified in Karlin et al., 1993, Proc. Natl. Acad. Sci., 90:5873-5877, and incorporated into the NBLAST and XBLAST programs (Altschul et al., 1991, Nucleic Acids Res., 25:3389-3402). In certain aspects, Gapped BLAST can be used as described in Altschul et al., 1997, Nucleic Acids Res. 25:3389-3402. BLAST-2, WU-BLAST-2 (Altschul et al., 1996, Methods in Enzymology, 266:460-480), ALIGN, ALIGN-2 (Genentech, South San Francisco, California) or Megalign (DNASTAR) are additional publicly available software programs that can be used to align sequences. In certain aspects, the percent identity between two nucleotide sequences is determined using the GAP program in the GCG software package (e.g., using a NWSgapdna.CMP matrix and a gap weight of 40, 50, 60, 70, or 90 and a length weight of 1, 2, 3, 4, 5, or 6). In certain alternative aspects, the GAP program in the GCG software package, which incorporates the algorithm of Needleman and Wunsch (J. Mol. Biol. (48):444-453 (1970)) can be used to determine the percent identity between two amino acid sequences (e.g., using either a BLOSUM 62 matrix or a PAM250 matrix, and a gap weight of 16, 14, 12, 10, 8, 6, or 4 and a length weight of 1, 2, 3, 4, 5). Alternatively, in certain aspects, the percent identity between nucleotide or amino acid sequences is determined using the algorithm of Myers and Miller (CABIOS, 4:11-17 (1989)). For example, the percent identity can be determined using the ALIGN program (version 2.0) and using a PAM120 with residue table, a gap length penalty of 12 and a gap penalty of 4. One skilled in the art can determine appropriate parameters for maximal alignment by particular alignment software. In certain aspects, the default parameters of the alignment software are used.

One skilled in the art will appreciate that the generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is ClustalW2, available from www.clustal.org. Another suitable program is MUSCLE, available from www.drive5.com/muscle/. ClustalW2 and MUSCLE are alternatively available, e.g., from the EBI (European Bioinformatics Institute). In certain aspects, the percentage identity “X” of a first nucleotide sequence to a second nucleotide sequence is calculated as 100×(Y/Z), where Y is the number of nucleotide residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence. Different regions within a single polynucleotide target sequence that align with a polynucleotide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

According to the present application, the degree of identity, between a given reference nucleotide sequence and a nucleotide sequence which is a homologue of said given nucleotide sequence will preferably be at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%. The degree of identity is given preferably for a nucleic acid region which is at least 10%, at least 20%, at least 30%, at least 40%, at least 50%, at least 60%, at least 70%, at least 80%, at least 90% or 100% of the entire length of the reference nucleic acid sequence. For example, if the reference nucleic acid sequence consists of 200 nucleotides, the degree of identity is given preferably for at least 20, at least 40, at least 60, at least 80, at least 100, at least 120, at least 140, at least 160, at least 180, or 200 nucleotides, preferably contiguous nucleotides. In a particular embodiment, the degree/percentage of similarity or identity is given for the entire length of the reference nucleic acid sequence.

The term “amino acid identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue (e.g., Ala, Pro, Ser, Thr, Gly, Val, Leu, lie, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. According to the present application, the degree of identity, between a given reference amino acid sequence and an amino acid sequence which is a homologue of said given amino acid sequence will preferably be at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%. The degree of identity is given preferably for an amino acid region which is at least 10%, at least 20%, at least 30%, at least 40%, at least 50%, at least 60%, at least 70%, at least 80%, at least 90% or 100% of the entire length of the reference amino acid sequence. For example, if the reference amino acid sequence consists of 200 amino acids, the degree of identity is given preferably for at least 20, at least 40, at least 60, at least 80, at least 100, at least 120, at least 140, at least 160, at least 180, or 200 amino acids, preferably contiguous amino acids. In a particular embodiment, the degree/percentage of similarity or identity is given for the entire length of the reference amino acid sequence.

“Homologue” or “homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived.

The term “defined by SEQ ID No. X” or “as depicted in SEQ ID No. X” as used herein refers to a biological sequence consisting of the sequence of amino acids or nucleotides given in the SEQ ID No. X. For instance, a protein defined in/by SEQ ID No. X consists of the amino acid sequence given in SEQ ID No. X. A further example is an amino acid sequence comprising SEQ ID No. X, which refers to an amino acid sequence longer than the amino acid sequence given in SEQ ID No. X but entirely comprising the amino acid sequence given in SEQ ID No. X (wherein the amino acid sequence given in SEQ ID No. X can be located N-terminally or C-terminally in the longer amino acid sequence, or can be embedded in the longer amino acid sequence), or to an amino acid sequence consisting of the amino acid sequence given in SEQ ID No. X.

The term “in vivo medical imaging” refers to the technique and process that is used to visualize the inside of an organism's body (or parts and/or functions thereof), for clinical purposes (e.g. disease diagnosis, prognosis or therapy monitoring) or medical science (e.g. study of anatomy and physiology). Examples of medical imaging methods include invasive techniques, such as intravascular ultrasound (IVUS), as well as non-invasive techniques, such as magnetic resonance imaging (MRI), ultrasound (US) and nuclear imaging. Examples of nuclear imaging include positron emission tomography (PET) and single photon emission computed tomography (SPECT). In a preferred embodiment, a nuclear imaging approach is used for in vivo medical imaging. According to one specific embodiment, in vivo pinhole SPECT/micro-CT (computed tomography) imaging is used as in vivo imaging approach.

As used herein, the term “radionuclide” relates to a radioactive label, which is a chemical compound in which one or more atoms have been replaced by a radioisotope. Radionuclides vary based on their characteristics, which include half-life, energy emission characteristics, and type of decay. This allows one to select radionuclides that have the desired mixture of characteristics suitable for use diagnostically and/or therapeutically. For example, gamma emitters are generally used diagnostically and alpha and beta emitters are generally used therapeutically. However, some radionuclides are both gamma emitters, alpha emitters and/or beta emitters, and thus, may be suitable for both uses. Radionuclides, as used herein, include for example—but not limited to—Actinium-225, Astatine-209, Astatine-210, Astatine-211, Bismuth-212, Bismuth-213, Brome-76, Caesium-137, Carbon-11, Chromium-51, Cobalt-60, Copper-64, Copper-67, Dysprosium-165, Erbium-169, Fermium-255, Fluorine-18, Gallium-67, Gallium-68, Gold-198, Holium-166, Indium-111, Iodine-123, Iodine-124, Iodine-125, Iodine-131, Iridium-192, Iron-59, Krypton-81m, Lead-212, Lutetium-177, Molydenum-99, Nitrogen-13, Oxygen-15, Palladium-103, Phosphorus-32, Potassium-42, Radium-223, Rhenium-186, Rhenium-188, Samarium-153, Technetium-99m, Radium-223, Rubidium-82, Ruthenium-106, Sodium-24, Strontium-89, Terbium-149, Thallium-201, Thorium-227, Xenon-133, Ytterbium-169, Ytterbium-177, Yttrium-86, Yttrium-90, Zirconium-89. In certain embodiments, the radionuclide is selected from the group of radionuclides as described above. In a specific embodiment, the radionuclide is selected from the group consisting of Technetium-99m, Gallium-68, Fluorine-18, Indium-111, Zirconium-89, Iodine-123, Iodine-124, Iodine-131, Astatine-211, Bismuth-213, Lutetium-177 and Yttrium-86.

A “patient” or “subject”, for the purpose of this application, relates to any organism such as a vertebrate, particularly any mammal, including both a human and another mammal, e.g., an animal such as a rodent, a rabbit, a cow, a sheep, a horse, a dog, a cat, a lama, a pig, or a non-human primate (e.g., a monkey). In one embodiment, the patient is a human, a rat or a non-human primate. Preferably, the patient is a human. In one embodiment, a patient is a subject with or suspected of having a disease or disorder, or an injury. In the context of this application, the disease is cancer, more particularly cancer characterised by TfR expressing tumor cells.

The terms “treatment” or “treating” or “treat” can be used interchangeably and are defined by a therapeutic intervention that slows, interrupts, arrests, controls, stops, reduces, or reverts the progression or severity of a sign, symptom, disorder, condition, injury, or disease, but does not necessarily involve a total elimination of all disease-related signs, symptoms, conditions, or disorders. Those in need of treatment include those already diagnosed with the disorder as well as those prone or predisposed to contract the disorder or those in whom the disorder is to be prevented. For example, in tumor (e.g. cancer) treatment, a therapeutic agent can directly decrease the pathology of tumor cells, or render the tumor cells more susceptible to treatment by other therapeutic agents or by the subject's own immune system.

As used herein, the term “therapeutically effective amount” means the amount needed to achieve the desired result or results when used in therapy.

As used herein, the terms “diagnosis”, “prognosis” and/or “prediction” comprise diagnosing, prognosing and/or predicting a certain disease and/or disorder, thereby predicting the onset and/or presence of a certain disease and/or disorder, and/or predicting the progress and/or duration of a certain disease and/or disorder, and/or predicting the response of a patient suffering αom a certain disease and/or disorder to therapy.

The term “statistically significantly” different is well known by the person skilled in the art. Statistical significance plays a pivotal role in statistical hypothesis testing. It is used to determine whether the null hypothesis should be rejected or retained. The null hypothesis is the default assumption that nothing happened or changed. For the null hypothesis to be rejected, an observed result has to be statistically significant, i.e. the observed p-value is less than the pre-specified significance level α. The p-value of a result, p, is the probability of obtaining a result at least as extreme, given that the null hypothesis were true. In one embodiment, α is 0.05. In a more particular embodiment, α is 0.01. In an even more particular embodiment, a is 0.001.

### Transferrin Receptor

The transferrin receptor is a cell surface receptor necessary for cellular iron uptake by the process of receptor-mediated endocytosis. This receptor is required for erythropoiesis and neurologic development.

Throughout current application, “transferrin receptor” or “TfR” are used interchangeably and refer to the human transferrin receptor as described above and depicted in SEQ ID No. 1, unless specified otherwise. The amino acid sequence of the Cynomolgus monkey transferrin receptor is depicted in SEQ ID No. 10.

(amino acid sequence of human TfR):
SEQ ID No. 1
MMDQARSAFS NLFGGEPLSY TRFSLARQVD GDNSHVEMKL AVDEEENADN NTKANVTKPK
RCSGSICYGT IAVIVFFLIG FMIGYLGYCK GVEPKTECER LAGTESPVRE EPGEDFPAAR
RLYWDDLKRK LSEKLDSTDF TGTIKLLNEN SYVPREAGSQ KDENLALYVE NQFREFKLSK
VWRDQHFVKI QVKDSAQNSV IIVDKNGRLV YLVENPGGYV AYSKAATVTG KLVHANFGTK
KDFEDLYTPV NGSIVIVRAG KITFAEKVAN AESLNAIGVL IYMDQTKFPI VNAELSFFGH
AHLGTGDPYT PGFPSFNHTQ FPPSRSSGLP NIPVQTISRA AAEKLFGNME GDCPSDWKTD
STCRMVTSES KNVKLTVSNV LKEIKILNIF GVIKGFVEPD HYVVVGAQRD AWGPGAAKSG
VGTALLLKLA QMFSDMVLKD GFQPSRSIIF ASWSAGDFGS VGATEWLEGY LSSLHLKAFT
YINLDKAVLG TSNFKVSASP LLYTLIEKTM QNVKHPVTGQ FLYQDSNWAS KVEKLTLDNA
AFPFLAYSGI PAVSFCFCED TDYPYLGTTM DTYKELIERI PELNKVARAA AEVAGQFVIK
LTHDVELNLD YERYNSQLLS FVRDLNQYRA DIKEMGLSLQ WLYSARGDFF RATSRLTTDF
GNAEKTDRFV MKKLNDRVMR VEYHFLSPYV SPKESPFRHV FWGSGSHTLP ALLENLKLRK
QNNGAFNETL FRNQLALATW TIQGAANALS GDVWDIDNEF
(amino acid sequence of Cynomolgus TfR):
SEQ ID No. 10
MMDQARSAFS NLFGGEPLSY TRFSLARQVD GDNSHVEMKL GVDEEENTDN NTKANGTKPK
RCGGNICYGT IAVIIFFLIG FMIGYLGYCK GVEPKTECER LAGTESPARE EPEEDFPAAP
RLYWDDLKRK LSEKLDTTDF TSTIKLLNEN LYVPREAGSQ KDENLALYIE NQFREFKLSK
VWRDQHFVKI QVKDSAQNSV IIVDKNGGLV YLVENPGGYV AYSKAATVTG KLVHANFGTK
KDFEDLDSPV NGSIVIVRAG KITFAEKVAN AESLNAIGVL IYMDQTKFPI VKADLSFFGH
AHLGTGDPYT PGFPSFNHTQ FPPSQSSGLP NIPVQTISRA AAEKLFGNME GDCPSDWKTD
STCKMVTSEN KSVKLTVSNV LKETKILNIF GVIKGFVEPD HYVVVGAQRD AWGPGAAKSS
VGTALLLKLA QMFSDMVLKD GFQPSRSIIF ASWSAGDFGS VGATEWLEGY LSSLHLKAFT
YINLDKAVLG TSNFKVSASP LLYTLIEKTM QDVKHPVTGR SLYQDSNWAS KVEKLTLDNA
AFPFLAYSGI PAVSFCFCED TDYPYLGTTM DTYKELVERI PELNKVARAA AEVAGQFVIK
LTHDTELNLD YERYNSQLLL FLRDLNQYRA DVKEMGLSLQ WLYSARGDFF RATSRLTTDF
RNAEKRDKFV MKKLNDRVMR VEYYFLSPYV SPKESPFRHV FWGSGSHTLS ALLESLKLRR
QNNSAFNETL FRNQLALATW TIQGAANALS GDVWDIDNEF

The current application provides antibodies and antibody fragments that bind the human transferrin receptor. The development of antibodies against the human TfR is part of a promising strategy for targeted treatment and immunotherapy.

### TfR Binding Agents

In a first aspect, the present application discloses binding agents, more particularly antibodies, even more particularly single variable domain antibodies, most particularly VHHs, that recognize and bind to the human and/or NHP transferrin receptor. These antibodies are thus TfR binding agents. In various embodiments, said TfR binding agents bind to, but do not functionally modulate iron transport. In other embodiments, said TfR binding agents are also able to detach from the TfR after binding to it. This is especially useful in the process of transferrin receptor mediated transcytosis, a process during which the transferrin receptor binds cargo at the peripheral side of for example the BBB endothelial cells, transports the cargo through said cells and sets the cargo free at the brain side of the BBB endothelial cells. The TfR binding agents of current application are thus extremely helpful in brain delivery of drugs which are directly or indirectly administered in peripheral blood. The TfR binding agents of current application are equally useful in delivery of therapeutic and/or imaging compounds to cancer cells. Therefore, the present application also provides compositions comprising TfR binding agents (see later). Said compositions can be pharmaceutical and/or imaging compositions and current application envisages their use in the treatment and/or study of various CNS diseases and/or TfR expressing cancers. In various embodiments, the TfR binding agents of the application comprise a targeting moiety having an antigen recognition domain that recognizes an epitope present on TfR. In an embodiment, the antigen-recognition domain recognizes one or more linear epitopes present on TfR. As used herein, a linear epitope refers to any continuous sequence of amino acids present on TfR. In another embodiment, the antigen-recognition domain recognizes one or more conformational epitopes present on TfR. As used herein, a conformation epitope refers to one or more sections of amino acids (which may be discontinuous) which form a three-dimensional surface with features and/or shapes and/or tertiary structures capable of being recognized by an antigen recognition domain.

In an embodiment, the TfR binding agent of the application comprises a targeting moiety with an antigen recognition domain that recognizes one or more epitopes present on the human TfR. In an embodiment, the human TfR comprises the amino acid sequence of SEQ ID No. 1. In a more particular embodiment, the human TfR consists of the amino acid sequence of SEQ ID No. 1. In an even more particular embodiment the TfR binding agents of the application do not compete with iron transport.

In one embodiment, the TfR binding agent of the application comprises a full-length multimeric protein that includes two heavy chains and two light chains. Each heavy chain includes one variable region (e.g. VH) and at least three constant regions (e.g. CH1, CH2 and CH3), and each light chain includes one variable region (VL) and one constant region (CL). As described above in the definitions section, the variable regions determine the specificity of the antibody and comprise three hypervariable regions also known as complementarity determining regions (CDRs) that contribute to the antibody binding specificity.

In some embodiments, the TfR binding agent comprises a targeting moiety which is an antibody fragment. The term “antibody fragment” refers to a portion of any antibody or antibody-like structure that by itself has high affinity for an antigenic determinant or epitope, and contains one or more CDRs accounting for such specificity. In some particular embodiments, the TfR binding agent of the application comprises a targeting moiety which is a single-domain antibody, an immunoglobulin single variable domain, a heavy-chain-only antibody (VHH), a single-chain antibody (scFv), a shark heavy-chain-only antibody (VNAR), a microprotein (cysteine knot protein, knottin), a DARPin, a Tetranectin, an Affibody, an Affimer, a Transbody, an Anticalin, an AdNectin, an Affilin, a Microbody, a peptide aptamer, an alterases, a plastic antibodies, a phylomer, a stradobodies, a maxibodies, an evibody, a fynomer, an armadillo repeat protein, a Kunitz domain, an avimer, an atrimer, a probody, an immunobody, a triomab, a troybody, a pepbody, a vaccibody, a UniBody, a DuoBody, a Fv, a Fab, a Fab′, a F(ab′)2, a peptide mimetic molecule, or a synthetic molecule, as described in US Patent Nos. or Patent Publication Nos. U.S. Pat. No. 7,417,130, US 2004/132094, U.S. Pat. No. 5,831,012, US 2004/023334, U.S. Pat. Nos. 7,250,297, 6,818,418, US 2004/209243, U.S. Pat. Nos. 7,838,629, 7,186,524, 6,004,746, 5,475,096, US 2004/146938, US 2004/157209, U.S. Pat. Nos. 6,994,982, 6,794,144, US 2010/239633, U.S. Pat. No. 7,803,907, US 2010/119446, and/or U.S. Pat. No. 7,166,697, the contents of which are hereby incorporated by reference in their entireties. See also Storz 2011 MAbs 3: 310-317.

In particular embodiments, the TfR binding agent of the application comprises a targeting moiety which is a single-domain antibody, such as a VHH. The VHH may be derived from, for example, an organism that produces VHH antibodies such as a camelid, a shark, or the VHH may be a designed VHH. VHHs are antibody-derived therapeutic proteins that contain the unique structural and functional properties of naturally occurring heavy-chain antibodies (see definition section above). In some embodiments, the single domain antibody as described herein is an immunoglobulin single variable domain or ISVD. In most particular embodiments, the TfR binding agent comprises a targeting moiety which is a VHH.

In a specific embodiment, the TfR binding agent of the application more particularly the ISVD or VHH of the application comprises a CDR3 set forth in SEQ ID No. 5 or SEQ ID No. 9 or having an amino acid sequence with maximally two amino acids different to SEQ ID No. 5 or SEQ ID No. 9 or with maximally one amino acid different to SEQ ID No. 5 or SEQ ID No. 9 or comprises a CDR3 comprising or consisting of the amino acid sequence depicted in SEQ ID No. 5 or SEQ ID No. 9. “Maximally two” means 0, 1 or 2.

Said CDR3 sequence represents an essential feature of a family of ISVDs, more particularly VHHs, specifically binding TfR at the same binding site. An ISVD family is defined herein as a group of ISVD amino acid sequences with high similarity, or even identical, in the CDR3 sequence. By default, ISVDs belong to the same family when binding to the same target epitope. Variations in an ISVD family may be interesting if expression/stability/affinity/crystallization of a representative of that family is poor, as small deviations like single amino acid mutations occurring within one family may improve these properties.

In yet another specific embodiment the TfR binding agent of the application more particularly the ISVD or VHH of the application comprises a CDR3 set forth in SEQ ID No. 5 or SEQ ID No. 9 or having an amino acid sequence with maximally two amino acids different to SEQ ID No. 5 or SEQ ID No. 9 or with maximally one amino acid different to SEQ ID No. 5 or SEQ ID No. 9 or comprises a CDR3 comprising or consisting of the amino acid sequence depicted in SEQ ID No. 5 or SEQ ID No. 9 and/or comprises a CDR2 set forth in SEQ ID No. 4 or SEQ ID No. 8 or having an amino acid sequence with maximally two amino acids different to SEQ ID No. 4 or SEQ ID No. 8 or with maximally one amino acid different to SEQ ID No. 4 or SEQ ID No. 8 or comprises a CDR3 comprising or consisting of the amino acid sequence depicted in SEQ ID No. 4 or SEQ ID No. 8 and/or comprises a CDR1 set forth in SEQ ID No. 3 or SEQ ID No. 7 or having an amino acid sequence with maximally two amino acids different to SEQ ID No. 3 or SEQ ID No. 7 or with maximally one amino acid different to SEQ ID No. 3 or SEQ ID No. 7 or comprises a CDR1 comprising or consisting of the amino acid sequence depicted in SEQ ID No. 3 or SEQ ID No. 7.

One embodiment relates to the ISVDs of the application comprising SEQ ID No. 2 or SEQ ID No. 6, or homologues thereof with at least 80%, 85%, 90%, 92%, 95%, 96%, 97%, 98% or 99% homology on amino acid level, or a humanized variant thereof.

In another embodiment the invention provides ISVDs comprising sequences depicted in SEQ ID No. 11, 12, 13, 14, . . . to SEQ ID No. 31 or sequences depicted in SEQ ID No. 37, 38, 39, . . . to SEQ ID No. 68.

In a specific embodiment the invention provides humanized variants of the ISVDs, depicted in SEQ ID No. 2 or SEQ ID No. 6, wherein said humanized variants are depicted in sequences SEQ ID No. 11 to SEQ ID No. 31.

In another specific embodiment the invention provides variants of the ISVDs, depicted in SEQ ID No. 2 or SEQ ID No. 6, wherein said variants have amino acid substitutions in the CDR1 and/or CDR2 and/or CDR3 to histidine amino acids and wherein said variants are depicted in sequences SEQ ID No. 37 to SEQ ID No. 68.

In another specific embodiment the invention provides variants of the ISVDs, depicted in SEQ ID No. 2 or SEQ ID No. 6, wherein said variants have amino acid substitutions in the CDR1 and/or CDR2 and/or CDR3 to histidine amino acids and wherein said variants are depicted in sequences SEQ ID No. 37 to SEQ ID No. 68 and wherein said variants are further humanized variants.

In another specific embodiment the invention provides variants of the ISVDs, depicted in SEQ ID No. 2 or SEQ ID No. 6, wherein said variants have amino acid substitutions in the CDR1 and/or CDR2 and/or CDR3 to histidine amino acids and wherein said variants are obtained by one amino acid substitution to histidine in CDR1 and CDR2 or CDR1 and CDR2 and CDR3 or CDR2 and CDR3 wherein said variants are combined from the variants depicted in sequences SEQ ID No. 37 to SEQ ID No. 68.

Table 1 provides an overview of full length and CDR sequences of the herein disclosed anti-TfR VHHs.

TABLE 1
Overview of full length and CDR sequences
of obtained TfR binding VHHs

| | Full | | | |
|---|---|---|---|---|
| VHH | length | CDR1 | CDR2 | CDR3 |
| BBB00533 | SEQ ID | SEQ ID No. 3 | SEQ ID No. 4 | SEQ ID No. 5 |
| | No. 2 | GRTFNYAMG | TIDWKDGSSY | GDGDYCSTYTCAAEVEYDY |
| BBB00515 | SEQ ID | SEQ ID No. 7 | SEQ ID No. 8 | SEQ ID No. 9 |
| | No. 6 | GSIFSINAMG | VITSGGSTI | HVGLKVPTIQELSLGFGS |

In some embodiments, the TfR binding agent of the application comprises a targeting moiety which is a VHH comprising a single amino acid chain having four “framework regions” and three “complementary determining regions” or CDRs. As used herein, “framework region” refers to a region in the variable domain which is located between the CDRs. As used herein, “complementary determining region” or “CDR” refers to variable regions in VHHs that contains the amino acid sequences capable of specifically binding to antigenic targets. In various embodiments, the TfR binding agent comprises a VHH having a variable domain comprising at least one CDR1, CDR2, and/or CDR3 sequence. In some embodiments, the CDR1 sequence is selected from SEQ ID No. 3 or 7. In some embodiments, the CDR2 sequence is selected from SEQ ID No. 4 or 8. In some embodiments, the CDR3 sequence is selected from SEQ ID No. 5 or 9.

In a particular embodiment, a TfR binding agent is provided, said agent has an amino acid sequence of at least 80%, at least 85%, at least 90%, at least 91%, at least 92%, at least 93%, at least 94%, at least 95%, at least 96%, at least 97%, at least 98%, at least 99% homology to SEQ ID No. 2, said agent comprising three complementarity determining regions (CDR1, CDR2 and CDR3), wherein CDR1 comprises or consists of SEQ ID No. 3, CDR2 comprises or consists of SEQ ID No. 4 and CDR3 comprises or consist of SEQ ID No. 5. In particular embodiments, said differences in amino acid sequence between said homologues and SEQ ID No. 2 are found in the framework regions. The role of framework regions in specific binding to the target is rather limited and variations in the framework sequences are allowed to obtain a similar efficacy of said ISVDs (see for instance De Groeve et al 2010J Nuclear Medicine 51:782; Saerens et al 2005 J Mol Biol 352:597-607). In particular embodiments, said differences in amino acid sequence have been introduced for example for humanization purposes (see below). In even more particular embodiments, said differences in amino acid sequences are limited to conserved amino acid substitutions (see below). In most particular embodiments, a TfR binding agent is provided wherein said TfR binding agent is represented by SEQ ID No. 2.

In a particular embodiment, a TfR binding agent is provided, said agent has an amino acid sequence of at least 80%, at least 85%, at least 90%, at least 91%, at least 92%, at least 93%, at least 94%, at least 95%, at least 96%, at least 97%, at least 98%, at least 99% homology to SEQ ID No. 6, said agent comprising three complementarity determining regions (CDR1, CDR2 and CDR3), wherein CDR1 comprises or consist of SEQ ID No. 7, CDR2 comprises or consist of SEQ ID No. 8 and CDR3 comprises or consist of SEQ ID No. 9. In particular embodiments, said differences in amino acid sequence between said homologues and SEQ ID No. 6 are found in the framework regions. In most particular embodiments, a TfR binding agent is provided wherein said TfR binding agent is represented by SEQ ID No. 6.

In a particular embodiment, a TfR binding agent is provided, said agent has an amino acid sequence of at least 80%, at least 85%, at least 90%, at least 91%, at least 92%, at least 93%, at least 94%, at least 95%, at least 96%, at least 97%, at least 98%, at least 99% homology to SEQ ID No. 6, said agent comprising three complementarity determining regions (CDR1, CDR2 and CDR3), wherein CDR1 comprises or consist of SEQ ID No. 7, CDR2 comprises or consist of SEQ ID No. 8 and CDR3 comprises or consist of SEQ ID No. 9. In particular embodiments, said differences in amino acid sequence between said homologues and SEQ ID No. 6 are found in the framework regions. In most particular embodiments, a TfR binding agent is provided wherein said TfR binding agent is represented by SEQ ID No. 6.

### Humanization

In one embodiment, the TfR binding agent of current application comprises an immunoglobulin single variable domain or a VHH that has been “humanized”, i.e. one or more amino acid residues in the amino acid sequence of the VHH obtained by immunization is replaced by one or more of the amino acid residues that occur at the corresponding position(s) in a VH domain from a conventional 4-chain antibody from a human being to increase the degree of sequence identity with the closest human germline sequence. Potentially useful humanizing substitutions can be ascertained by comparing the sequence of the framework regions of a naturally occurring VHH sequence with the corresponding framework sequence of one or more closely related human VH sequence(s), after which one or more of the potentially useful humanizing substitutions (or combinations thereof) thus determined can be introduced into said VHH sequence (in any manner known per se, as further described herein) and the resulting humanized VHH sequences can be tested for affinity for the target, for stability, for ease and level of expression, and/or for other desired properties. In this way, by means of a limited degree of trial and error, other suitable humanizing substitutions (or suitable combinations thereof) can be determined by the skilled person. Also, based on what is described before, (the framework regions of) an immunoglobulin single variable domain, such as a VHH domain may be partially humanized or fully humanized.

Therefore, in various embodiments, the TfR binding agents of the application comprise a targeting moiety comprising an amino acid sequence having one or more amino acid mutations with respect to SEQ ID No. 2. In various embodiments, the TfR binding agent comprises a targeting moiety comprising an amino acid sequence having one, or two, or three, or four, or five, or six, or seven, or eight, or nine, or ten, or fifteen, or twenty amino acid mutations with respect to SEQ ID No. 2. In some embodiments, the one or more amino acid mutations may be independently selected from substitutions, insertions, deletions, and truncations. In some embodiments, the amino acid mutations are amino acid substitutions, and may include conservative and/or non-conservative substitutions. In particular embodiments, the one or more amino acid mutations may be in the CDRs of the targeting moiety (e.g., the CDR1, CDR2 or CDR3 regions). In other particular embodiments, the one or more amino acid mutations may be in the framework regions of the targeting moiety (e.g., the FR1, FR2, FR3, or FR4 regions). In most particular embodiments, said one or more amino acid mutations are only present in the framework regions of said TfR binding agents.

“Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe. As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices.

As used herein, “non-conservative substitutions” are defined as exchanges of an amino acid by another amino acid listed in a different group of the six standard amino acid groups (1) to (6) shown above.

In various embodiments, the substitutions may also include non-classical amino acids (e.g. selenocysteine, pyrrolysine, N-formylmethionine β-alanine, GABA and δ-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, δ-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general).

Humanization can be performed using humanization techniques known in the art. In some embodiments, possible humanizing substitutions or combinations of humanizing substitutions may be determined by methods known in the art, for example without the purpose of being limiting, by a comparison between the sequence of a VHH and the sequence of a naturally occurring human VH domain. In some embodiments, the humanizing substitutions are chosen such that the resulting humanized VHHs still retain advantageous functional properties. Generally, as a result of humanization, the VHHs of the application may become more “human-like”, while still retaining favourable properties such as a reduced immunogenicity, compared to the corresponding naturally occurring VHH domains. In various embodiments, the humanized VHHs of the application can be obtained in any suitable manner known in the art and thus are not strictly limited to polypeptides that have been obtained using a polypeptide that comprises a naturally occurring VHH domain as a starting material. Indeed, modification of the amino acid sequences may be achieved using any known technique in the art e.g., site-directed mutagenesis or PCR based mutagenesis. Such techniques are described, for example, in Sambrook et al., Molecular Cloning: A Laboratory Manual, Cold Spring Harbor Press, Plainview, N.Y., 1989 and Ausubel et al., Current Protocols in Molecular Biology, John Wiley & Sons, New York, N.Y., 1989.

In various embodiments, the mutations that were introduced for example to humanize the TfR binding agent do not substantially reduce the present TfR binding agent's capability to specifically bind to the human TfR. In various embodiments, the mutations do not substantially reduce the present TfR binding agent's capability to specifically bind to TfR without neutralizing TfR.

### Association Kinetics of the TfR Binding Agents

In various embodiments, the binding affinity of the TfR binding agent of the application for the full-length and/or mature forms and/or isoforms and/or splice variants and/or fragments and/or monomeric and/or dimeric and/or tetrameric forms and/or any other naturally occurring or synthetic analogs, variants, or mutants (including monomeric and/or dimeric and/or tetrameric forms) of human TfR may be described by the equilibrium dissociation constant (KD), alternatively by the dissociation constant koff. In various embodiments, the TfR binding agent comprises a targeting moiety that binds to the full-length and/or mature forms and/or isoforms and/or splice variants and/or fragments and/or any other naturally occurring or synthetic analogs, variants, or mutants (including monomeric and/or dimeric and/or tetrameric forms) of human TfR with a KD of less than 10 μM or more particularly of less than 1 μM and/or more than 1 nM. In other embodiments, the TfR binding agent of current application comprises a targeting moiety that binds to the full-length and/or mature forms and/or isoforms and/or splice variants and/or fragments and/or any other naturally occurring or synthetic analogues, variants, or mutants (including monomeric and/or dimeric and/or tetrameric forms) of human TfR with a KD between 1 nM and 1 μM or between 5 nM and 950 nM or between 10 nM and 900 nM or between 20 nM and 850 nM or between 30 nM and 800 nM or between 40 nM and 700 nM or between 50 nM and 600 nM or between 50 nM and 500 nM. In a more particular embodiment said KD for human TfR is between 1 nM and 100 nM or between 2 nM and 75 nM or between 3 nM and 50 nM or between 4 nM and 40 nM or between 5 nM and 30 nM or between 6 nM and 25 nM or between 7 nM and 20 nM. In other embodiments, the TfR binding agent comprises a targeting moiety that binds to the full-length and/or mature forms and/or isoforms and/or splice variants and/or fragments and/or any other naturally occurring or synthetic analogs, variants, or mutants (including monomeric and/or dimeric and/or tetrameric forms) of human TfR with a KD of about 300 nM, about 250 nM, about 275 nM, about 100 nM, about 75 nM, about 50 nM, about 25 nM or about 15 nM.

According to another embodiment of the application, the TfR binding agent of current application has an affinity for human and NHP TfR in the range from about 1 nM to about 1 μM, or in the range from about 2 nM to about 700 nM, or in the range from about 2 nM to about 60 nM or in the range from about 20 nM to 300 nM, e.g. as measured by biolayer interferometry (BLI) and/or ELISA.

In various embodiments, the ISVDs or VHHs of the application are not limited to a specific biological source or to a specific method of preparation. Said ISDV or VHH sequences can generally be generated or obtained by suitably immunizing a species of Camelid with a human and/or NHP TfR molecule (i.e. so as to raise an immune response and/or heavy chain antibodies directed against TfR), by obtaining a suitable biological sample from the Camelid (such as a blood sample, or any sample of B-cells), and by generating VHH sequences directed against TfR, starting from the sample, using any suitable known technique. VHHs can also be obtained by expressing a nucleotide sequence encoding a naturally occurring VHH domain, by “humanization” of a naturally occurring VHH domain or by expression of a nucleic acid encoding such humanized VHH domain, by using synthetic or semi-synthetic techniques for preparing proteins, polypeptides or other amino acid sequences known in the art, by preparing a nucleic acid encoding a VHH using techniques for nucleic acid synthesis known in the art, followed by expression of the nucleic acid thus obtained, and/or by any combination of one or more of the foregoing.

### Production of Transferrin Receptor Binding Agents

The TfR binding agents, particularly TfR antibodies, more particularly the ISVDs or VHHs of the application are not limited to a specific biological source or to a specific method of preparation. Methods for producing the TfR binding agents of the application are described herein. For example, DNA sequences encoding the TfR binding agents of the application can be easily prepared by the art-known techniques such as cloning, hybridization screening and Polymerase Chain Reaction (PCR). Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al. (1989), Maniatis et al. (1982), Wu (ed.) (1993) and Ausubel et al. (1992). Alternatively, DNA sequences encoding the TfR binding agents of the application can be chemically synthesized using methods known in the art. Synthetic DNA sequences can be ligated to other appropriate nucleotide sequences, including for example expression control sequences, to produce gene expression constructs encoding the desired TfR binding agents.

Accordingly, in various embodiments, the present application provides for isolated nucleic acids comprising a nucleotide sequence encoding the TfR binding agents described in current application. One embodiment further discloses an expression cassette comprising said nucleic acid molecule. More specific embodiments disclose the expression cassette wherein elements for cell- or tissue-specific expression are present. Further embodiments relate to a vector comprising said expression cassette or said nucleic acid molecule. More particular, said vector may be a viral vector, even more particular a lentiviral or AAV vector.

In order to produce the TfR binding agents of current application, expression vectors comprising a nucleic acid sequence encoding said TfR binding agents can then be introduced into host cells through transfection, transformation, or transduction techniques. Hence, in various embodiments, the present application provides for a host cell comprising a nucleic acid encoding one of the TfR binding agents of the present application. For example, nucleic acids encoding the TfR binding agent of the application can be introduced into host cells by retroviral transduction. Illustrative host cells are E. coli cells, Chinese hamster ovary (CHO) cells, yeast cells such as Pichia spp., human embryonic kidney 293 (HEK 293) cells, HeLa cells, baby hamster kidney (BHK) cells, monkey kidney cells (COS), human hepatocellular carcinoma cells (e.g., Hep G2), and myeloma cells. Transformed host cells can be grown under conditions that permit the host cells to express the genes that encode the TfR binding agent of the application.

Following expression, the TfR binding agents can be harvested and purified using techniques well known in the art, e.g. affinity tags such as glutathione-S-transferase (GST) and histidine (His) tags or by chromatography. Specific expression and purification conditions will vary depending upon the expression system employed. For example, if a gene is to be expressed in E. coli, it is first cloned into an expression vector by positioning the engineered gene downstream from a suitable bacterial promoter, e.g. Trp or Tac, and a prokaryotic signal sequence. In another example, if the engineered gene is to be expressed in eukaryotic host cells, e.g. CHO cells, it is first inserted into an expression vector containing for example, a suitable eukaryotic promoter, a secretion signal, enhancers, and various introns. In an embodiment, the TfR binding agent of the application comprises a His tag, a FLAG-tag and/or a Myc tag. In an embodiment, the TfR binding agent of the application comprises a His tag and a proteolytic site to allow cleavage of the His tag.

Current application thus also provides a host cell comprising one of the TfR binding agents described herein. Host cells comprising one of the nucleic acid molecules or the expression cassettes or the vectors of the application are provided herein as well. Host cells can be either prokaryotic or eukaryotic. Representative host cells that may be used with the invention include, but are not limited to, bacterial cells, yeast cells, plant cells and animal cells. Bacterial host cells suitable for use with the invention include Escherichia spp. cells, Bacillus spp. cells, Streptomyces spp. cells, Erwinia spp. cells, Klebsiella spp. cells, Serratia spp. cells, Pseudomonas spp. cells, and Salmonella spp. cells. Yeast host cells suitable for use with the invention include species within Saccharomyces, Schizosaccharomyces, Kluyveromyces, Pichia (e.g. Pichia pastoris), Hansenula (e.g. Hansenula polymorpha), Yarrowia, Schwaniomyces, Schizosaccharomyces, Zygosaccharomyces and the like. Saccharomyces cerevisiae, S. carlsbergensis and K. lactis are the most commonly used yeast hosts and are convenient fungal hosts. Animal host cells suitable for use with the invention include insect cells and mammalian cells (most particularly derived from Chinese hamster (e.g. CHO), and human cell lines, such as HeLa). Exemplary insect cell lines include, but are not limited to, Sf9 cells, baculovirus-insect cell systems (e.g. review Jarvis 2003 Virology 310:1-7). Non-limiting examples of plant cells include tobacco cells, Arabidopsis cells, tomato cells, maize cells, algae cells, among others. The host cells may be provided in suspension or flask cultures, tissue cultures, organ cultures and the like. Alternatively, the host cells may also be transgenic animals.

Animal or mammalian host cells suitable for harboring, expressing, and producing one of the TfR binding agents of the application include Chinese hamster ovary cells (CHO), such as CHO-K1 (ATCC CCL-61), DG44 (Chasin et al 1986 Som Cell Mol Genet 12:555-556; Kolkekar et al 1997 Biochemistry 36:10901-10909), CHO-K1 Tet-On cell line (Clontech), CHO designated ECACC 85050302 (CAMR, Salisbury, Wiltshire, UK), CHO clone 13 (GEIMG, Genova, IT), CHO clone B (GEIMG, Genova, IT), CHO-K1/SF designated ECACC 93061607 (CAMR, Salisbury, Wiltshire, UK), RR-CHOK1 designated ECACC 92052129 (CAMR, Salisbury, Wiltshire, UK), dihydrofolate reductase negative CHO cells (CHO/-DHFR, Urlaub & Chasin 1980 PNAS 77:4216), and dp12.CHO cells (U.S. Pat. No. 5,721,121); monkey kidney CV1 cells transformed by SV40 (COS cells, COS-7, ATCC CRL-1651); human embryonic kidney cells (e.g., 293 cells, or 293T cells, or 293 cells subcloned for growth in suspension culture, Graham et al 1977 J Gen Virol 36:59, or GnTI KO HEK293S cells, Reeves et al 2002 PNAS 99:13419); baby hamster kidney cells (BHK, ATCC CCL-10); monkey kidney cells (CV1, ATCC CCL-70); African green monkey kidney cells (VERO-76, ATCC CRL-1587; VERO, ATCC CCL-81); mouse sertoli cells (TM4, Mather 1980 Biol Reprod 23:243-251); human cervical carcinoma cells (HELA, ATCC CCL-2); canine kidney cells (MDCK, ATCC CCL-34); human lung cells (W138, ATCC CCL-75); human hepatoma cells (HEP-G2, HB 8065); mouse mammary tumor cells (MMT 060562, ATCC CCL-51); buffalo rat liver cells (BRL 3A, ATCC CRL-1442); TRI cells (Mather, 1982, Annals NYAcad. Sci., 383:44-68); MCR 5 cells; FS4 cells. According to a particular embodiment, the cells are mammalian cells selected from Hek293 cells or COS cells.

The host cells described above can be transiently or stably transfected. Such transfection of DNA, such as nucleic acid molecules, expression cassettes or expression vectors into prokaryotic and eukaryotic cells can be accomplished via any technique known in the art, including but not limited to standard bacterial transformations, calcium phosphate co-precipitation, electroporation, or liposome mediated-, DEAE dextran mediated-, polycationic mediated-, or viral mediated transfection. For all standard techniques see, for example, Molecular Cloning: A Laboratory Manual (Sambrook, et al., 1989, Cold Spring Harbor Laboratory Press; Culture of Animal Cells: A Manual of Basic Technique, 2* Ed. (R.I. Freshney. 1987. Liss, Inc. New York, N.Y.). The host cell may also be a recombinant host cell, which involves a cell which has been genetically modified to contain an isolated DNA molecule, nucleic acid molecule or expression construct or vector of the invention. The DNA can be introduced by any means known to the art which are appropriate for the particular type of cell, including without limitation, transformation, lipofection, electroporation or viral mediated transduction.

Furthermore, in alternative embodiments, the use of the herein described nucleic acid molecules, expression cassettes, or vectors encoding the TfR binding agents, particularly TfR antibodies, more particularly TfR ISVDs or VHHs is provided for the production of said TfR binding agent, antibodies, ISVDs or VHHs. In a particular embodiment, said use is provided for production of an intrabody. An intracellular antibody or “intrabody” is an antibody or a fragment of an antibody that is heterologously expressed within a designated intracellular compartment, a process which is made possible through the in-frame incorporation of intracellular trafficking signals. An intrabody can be expressed in any shape or form such as an intact IgG molecule or a Fab fragment, more particularly as genetically engineered antibody fragment for example as single domain intrabodies or VHHs. For a review see Zhu, and Marasco, 2008 (Therapeutic Antibodies. Handbook of Experimental Pharmacology 181. Ed. Springer-Verlag Berlin Heidelberg).

### Chimeras and Fusions

In various embodiments, any of the TfR binding agents of the application is provided as part of a chimera or fusion with one or more other agents. In particular embodiments, said other agent is a cytotoxic agent, a therapeutic agent, an imaging agent, radionuclide, an antisense oligonucleotide, an interfering RNA, an antibody or antibody fragment including another VHH. In other particular embodiments, said other agent is a nanoparticle, a lipid nanoparticle or an exosome. Alternatively phrased, a composition, more particularly a pharmaceutical composition is provided comprising any of the TfR binding agents of current application coupled to one or more other agents. In one embodiment, said agent is a chemical entity. The term “chemical entity” as used herein refers to simple or complex organic and inorganic molecules. Non-limiting examples of a chemical entity as used in current application is a peptide, peptidomimetic, protein, antibody (incl. antibody fragments such as ISVDs and VHHs), carbohydrate, nucleic acid or derivative thereof, a ligand, a substrate, a phosphate, an agonist, an antagonist, a neurotransmitter, an inhibitor, a drug. In one embodiment, said chemical entity is a biological, a small molecule, a therapeutic agent, an imaging agent or a test compound.

“Biological” as used here refers to a substance that is made from a living organism or its products. A biological can be composed of sugars, proteins, or nucleic acids or complex combinations of these substances, or may be living entities such as cells and tissues. Biologics are isolated from a variety of natural sources—human, animal, or microorganism—and may be produced by biotechnology methods and other cutting-edge technologies. A non-limiting example of a biological is an antibody.

A “small molecule” as used herein (as in the field of molecular biology and pharmacology) refers to a low molecular weight (<900 daltons) organic compound that may regulate a biological process. Most drugs are small molecules. Larger structures such as nucleic acids and proteins, and many polysaccharides are not small molecules, although their constituent monomers (ribo- or deoxyribonucleotides, amino acids, and monosaccharides, respectively) are considered small molecules. Small molecules can have a variety of biological functions or applications, serving as cell signalling molecules, drugs in medicine, pesticides in farming, and in many other roles for example by inhibiting a specific function of a protein or disrupt protein-protein interactions. These compounds can be natural (such as secondary metabolites) or artificial (such as peptidomimetics).

A “therapeutic agent” as used herein refers to a substance capable of slowing, interrupting, arresting, controlling, stopping, reducing or reverting the progression or severity of a sign, symptom, disorder, condition, injury, or disease, but does not necessarily involve a total elimination of all disease-related signs, symptoms, conditions, or disorders. Non-limiting examples of therapeutic agents are pharmaceutical agents, antibodies, antibody fragments, enzymes, antibiotics, antiproliferative agents, hormones, neurotransmitters, small molecules.

An “imaging agent” is a compound that has one or more properties that permit its presence and/or location to be detected directly or indirectly. Examples of such imaging agents include proteins and small molecule compounds incorporating a labelled moiety that permits detection, e.g. fluorescence or radioactivity.

The term “test compound” is used herein in the context of a “drug candidate compound” or a “candidate compound for lead optimization” in therapeutics, described in connection with the methods of the present invention. A “test compound” is thus not used as such in commercial settings but that can be used for lead optimization. These compounds comprise organic or inorganic compounds, derived synthetically or from natural resources. The compounds include polynucleotides, lipids or hormone analogues that are characterized by low molecular weights. Other biopolymeric organic test compounds include small peptides or peptide-like molecules (peptidomimetics) comprising from about 2 to about 40 amino acids and larger polypeptides comprising from about 40 to about 500 amino acids, such as antibodies or antibody conjugates.

The above described coupling between a TfR binding agent of the application and said compound can be achieved by chemical cross-linkers or by generating fusion proteins. Covalent conjugation can either be direct or via a linker. In certain embodiments, direct conjugation is by construction of a protein fusion (i.e. by genetic fusion of two or more genes—encoding one of the TfR binding agents of the application and one or more other proteins—and expression as a single protein). In certain embodiments, direct conjugation is by formation of a covalent bond between a reactive group on one or more portions of the TfR binding agent of the application and a corresponding group or acceptor on the chemical entity (e.g. a neurological drug). In certain embodiments, direct conjugation is by modification (i.e. genetic modification) of one of the two molecules to be conjugated to include a reactive group (as non-limiting examples, a sulfhydryl group or a carboxyl group) that forms a covalent attachment to the other molecule to be conjugated under appropriate conditions. As one non-limiting example, a molecule (i.e. an amino acid) with a desired reactive group (i.e. a cysteine residue) may be introduced into, e.g. the TfR antibody and a disulfide bond formed with the chemical entity (e.g. neurological drug). Methods for covalent conjugation of nucleic acids to proteins are also known in the art (i.e. photocrosslinking, see e.g. Zatsepin et al 2005 Russ Chem Rev 74:77-95). Non-covalent conjugation can be by any non-covalent attachment means, including hydrophobic bonds, ionic bonds, electrostatic interactions, and the like, as will be readily understood by one of ordinary skill in the art. Conjugation may also be performed using a variety of linkers. For example, an TfR antibody and a neurological drug may be conjugated using a variety of bifunctional protein coupling agents such as Nsuccinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Peptide linkers, comprised of from one to twenty amino acids joined by peptide bonds, may also be used. In certain such embodiments, the amino acids are selected from the twenty naturally occurring amino acids. In certain other such embodiments, one or more of the amino acids are selected from glycine, alanine, proline, asparagine, glutamine and lysine. The linker may be a “cleavable linker” facilitating release of the chemical entity, for example upon delivery of a neurological drug to the brain or upon delivery of a therapeutic drug to a cancer cell. An acid-labile linker, peptidase-sensitive linker, photolabile linker, dimethyl linker or disulfide-containing linker (Chari et al., Cancer Res. 52:127-131 (1992); U.S. Pat. No. 5,208,020) are non-limiting examples that may be used.

According to one particular embodiment, the “coupling” can be achieved by generating a multi-specific antibody (e.g. a bispecific antibody). Multi-specific antibodies are monoclonal antibodies that have binding specificities for at least two different sites. In one embodiment, the multi-specific antibody comprises a first antigen binding site which binds the human and/or NHP TfR and a second antigen binding site. In one embodiment, said second antigen binding site is an antigen, more particularly a brain antigen selected from the list consisting of beta-secretase 1 (BACE1), amyloid beta, epidermal growth factor receptor (EGFR), human epidermal growth factor receptor 2 (HER2), Tau, apolipoprotein E4 (ApoE4), alpha-synuclein, CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), TREM2 and caspase 6. In another embodiment, said second antigen binding site is a cancer antigen. A “cancer antigen” or “tumor antigen” refers to an antigenic substance produced in cancer or tumor cells, i.e. it triggers an immune response in the host. Tumor antigens are useful tumor markers in identifying tumor cells with diagnostic tests and are potential candidates for use in cancer therapy. Non-limiting examples of cancer antigens are MAGE-1, NY-ESO-1a and BAGE (see Renkvist et al 2001 Cancer Immunology).

### Therapeutic and Diagnostic Application of the TfR Binding Agents of Current Application

### A Blood Brain Barrier Shuttle: TfR Binding Agents as BBB Transporting Agents

Hence, in one aspect of current application, any of the TfR binding agents of current application is provided for use in transporting a chemical entity across the blood brain barrier or for use in transporting a chemical entity to the brain. In line hereby, the use is provided of the TfR binding agents of current application to transport a chemical entity across the blood brain barrier or to the brain. Also the use is provided of the TfR binding agents of current application to facilitate, enable, increase or improve the CNS uptake of a chemical entity across the blood brain barrier. Uptake is improved or increased when said chemical entity is statistically significantly more abundant or at least 10%, 15%, 20%, 25%, 50%, 75%, 100% or at least 2-fold, 5-fold, 10-fold, 20-fold, 50-fold or 100-fold more abundant in the brain in the situation that said chemical entity is coupled to one of the TfR binding agents of current application compared to the situation that said chemical entity is not coupled to one of the TfR binding agents of current application.

The TfR binding agents of current application are also provided for use as a medicament, for use in in vivo medical imaging and for use to treat a neurological disease, neuropathic pain or cancer, particularly TfR expressing cancers or to prevent brain damage after brain injury.

In another particular embodiment, the TfR binding agent is capable of cross reacting with human and non-human primate TfR.

The TfR binding agent is also provided when coupled to a chemical entity to facilitate the uptake of the chemical entity into the central nervous system (CNS) across the blood brain barrier (BBB). Said chemical entity can be a biological, small molecule, therapeutic agent, a radionuclide, an antisense oligonucleotide, imaging agent or test compound. In a particular embodiment, said chemical entity is neurotensin or a neurotensin analogue. In a most particular embodiment, said TfR binding agent comprises or consists of an antibody or an antibody fragment, more particularly an immunoglobulin single variable domain or VHH.

Having demonstrated the in vivo BBB shuttle capacities of the antibodies herein disclosed, the application provides a novel human blood brain barrier shuttle. Said shuttle efficiently delivers a chemical entity to the brain, more particularly to the CNS. Alternatively phrased, the application provides a TfR binding agent suitable for delivery of a chemical entity to the brain, said binding agent is one of the TfR binding agent disclosed in current application. The transport of the chemical entity to the brain is significantly increased when comparing the transport of the chemical entity without being part of the shuttle or without being to the TfR binding agent of the application. In particular embodiments, said chemical entity is a neurological disorder drug.

Said blood brain barrier shuttle comprises an TfR binding agent comprising a CDR3 sequence with maximally two amino acids different to SEQ ID No. 5 or 9, or with maximally one amino acid different to SEQ ID No. 5 or 9, or as depicted in SEQ ID No. 5 or 9, wherein the shuttle has a dissociation constant koff for human TfR of less than 5×10⁻²/s, more particularly less than 4×10⁻², 3.5×10⁻², 3×10⁻², 2.9×10⁻², 2.8×10⁻², 2.7×10⁻², 2.6×10⁻², 2.5×10⁻², 2.4×10⁻², 2.3×10⁻², 2.2×10⁻², 2.15×10⁻², 2.1×10⁻², 2×10⁻², 1.9×10⁻², 1.8×10⁻², 1.7×10⁻², 1.6×10⁻², 1.5×10⁻², 1.4×10⁻², 1.3×10⁻², 1.2×10⁻², 1.1×10⁻², 1×10⁻², 9×10⁻³, or 8×10⁻³/s as determined by BLI. In one embodiment, the shuttle's koff for human TfR is between 3×10⁻⁴/s and 3×10⁻²/s, or between 5×10⁻⁴/s and 2×10⁻²/s or between 8×10⁻⁴ and 1×10⁻²/s, between 9×10⁻⁴ and 9×10⁻³/s or between 1×10⁻³ and 8×10⁻³/s as determined by BLI. Besides the TfR binding agent, the shuttle comprises a molecule or a moiety that is to be transported to the CNS, more particularly across the BBB. In one embodiment said TfR binding agent comprises a CDR2 sequence with maximally two amino acids different to SEQ ID No. 4 or 8, or with maximally one amino acid different to SEQ ID No. 4 or 8, or as depicted in SEQ ID No. 4 and 8 and/or a CDR1 sequence with maximally two amino acids different to SEQ ID No. 3 or 7 or with maximally one amino acid different to SEQ ID No. 3 or 7, or as depicted in SEQ ID No. 3 or 7, or more particular comprises or consists of the amino acid sequence with at least 90%, at least 91%, at least 92%, at least 93%, at least 94%, at least 95%, at least 96%, at least 97%, at least 98%, at least 99% or 100% identity over the full length of said sequence to SEQ ID No. 2 or 6. In one particular embodiment, the differences in amino acid sequence are found outside the CDR regions. The molecule or moiety which is part of the blood brain barrier shuttle can be a neurological disorder drug, an imaging compound, a nanoparticle or an exosome.

The blood brain barrier shuttle as described above can alternatively be phrased as a blood CNS barrier shuttle, a composition or a pharmaceutical composition, or more particularly a BBB shuttle.

In another aspect, the blood brain barrier shuttle, blood CNS barrier shuttle, BBB shuttle, said composition or said pharmaceutical composition is provided for use as medicament, more particular for use in the treatment or diagnosis of a neurological disorder. In one embodiment, the shuttle or composition comprises besides any of the above described TfR binding agents a neurological disorder drug, a cancer drug, a nanoparticle or an imaging compound. In a particular embodiment, the neurological disorder drug of the shuttle or composition is a biological, a small molecule, a therapeutic agent, a radionuclide, an antisense oligonucleotide or test compound.

In another particular embodiment, the composition or the shuttle is a multispecific antibody comprising said the human TfR binding agent as described above and a second antigen binding site which binds a brain antigen. Non-limiting examples of a brain antigen are beta-secretase 1 (BACE1), amyloid beta, epidermal growth factor receptor (EGFR), human epidermal growth factor receptor 2 (HER2), Tau, apolipoprotein E4 (ApoE4), alpha-synuclein, CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR) and caspase 6. The multispecific antibody can also comprise a second or further antigen binding site which binds a tumor or cancer antigen. Said multispecific antibody is especially advantageous in the treatment and/or diagnosis of brain cancers.

In one particular embodiment, the molecule or moiety that is part of the above-described shuttle or composition is neurotensin or a neurotensin agonist. The neurotensin body temperature assay is used as an elegant system to evaluate the activity of antibodies to cross the BBB. However, the VHH-Neurotensin fusion described herein has clinical relevance as well. First, studies show that there is a potential therapeutic use for neurotensin (or neurotensin agonist) induced hypothermia. Choi et al (2012 FASEB J 26:2799-2810) showed that administration of the NT agonist ABS-201 immediately or up to 60 min after stroke attack significantly reduced infarct formation and brain cell death in an animal model of focal ischemia and was effective in promoting long-term functional recovery in post-stroke animals. Similar studies on regulated hypothermia induced by NT agonists reduce oxidative stress in the brain during reperfusion from asphyxia cardiac arrest (Katz et al 2004 Brain Res 1017:85-91). Also, lowering body temperature with neurotensin or with a NT agonist provided a better neurologic outcome than brief external cooling in a rat model of near drowning (Katz et al 2004 Crit Care Med 32:806-810). Therefore, the shuttle or composition as described above with a koff of between 8×10⁻⁴ and 4×10⁻²/s or between 9×10⁻⁴/s and 3×10⁻²/s or between 1×10⁻³ and 2.5×10⁻²/s or between 1×10⁻³ and 2×10⁻²/s or between 3×10⁻⁴/s and 3×10⁻²/s, or between 5×10⁻⁴/s and 2×10⁻²/s or between 8×10⁻⁴ and 1×10⁻²/s, between 9×10⁻⁴ and 9×10⁻³/s or between 1×10⁻³ and 8×10⁻³/s for human TfR as determined by BLI is also provided for treating or preventing stroke, brain cell death after stroke or brain damage after brain injury. In a particular embodiment, the shuttle or composition comprises neurotensin or a neurotensin agonist.

Besides its potency in inducing hypothermia, Nemeroff et al (1979 PNAS 76:5368-5371) demonstrated that neurotensin is an important modulator of nociceptive transmission and on a molar basis is even more potent than morphine as an antinociceptive agent. Neurotensin provides strong analgesia when administered directly into the brain and reverses pain behaviour induced by the development of neuropathic and bone cancer pain in animal models (Demeule et al 2014 JIC 124:1199-1213). Neurotensin as part of a brain penetrable neurotherapeutic (e.g. by coupling to one of the TfR binding agents of current application) is effective for clinical management of persistent and chronic pain. Therefore, the shuttle or composition as described herein with a koff of between 8×10⁻⁴ and 4×10⁻²/s or between 9×10⁻⁴/s and 3×10⁻²/s or between 1×10⁻³ and 2.5×10⁻²/s or between 1×10⁻³ and 2×10⁻²/s or between 3×10⁻⁴/s and 3×10⁻²/s, or between 5×10⁻⁴/s and 2×10⁻²/s or between 8×10⁻⁴ and 1×10⁻²/s, between 9×10⁻⁴ and 9×10⁻³/s or between 1×10⁻³ and 8×10⁻³/s for human TfR as determined by BLI and comprising neurotensin or a neurotensin agonist is provided for use in the treatment of neuropathic pain.

A “neurological disorder” as used herein refers to a disease or disorder which affects the central nervous system or CNS and/or which has an etiology in the CNS. The “central nervous system” or “CNS” refers to the complex of nerve tissues that control physical function, and includes the brain and spinal cord. Exemplary CNS diseases or disorders include, but are not limited to neurodegenerative diseases (including, but not limited to Lewy body disease, Parkinson's disease, tauopathies (including, but not limited to Alzheimer's disease and supranuclear palsy)), post-poliomyelitis syndrome, Shy-Draeger syndrome, olivopontocerebellar atrophy, multiple system atrophy, striatonigral degeneration, prion diseases (including, but not limited to bovine spongiform encephalopathy, scrapie, Creutzfeldt-Jakob syndrome, kuru, Gerstmann-Straussler-Scheinker disease, chronic wasting disease and fatal familial insomnia), bulbar palsy, dystonia (including but not limited to DYT1 dystonia), motor neuron diseases (including but not limited to multiple sclerosis, Charcot-Marie-Tooth (CMT) disease, amyotrophic lateral sclerosis (ALS)), and nervous system heterodegenerative disorders (including, but not limited to Canavan disease, Huntington's disease, neuronal ceroid-lipofuscinosis, Alexander's disease, Tourette's syndrome, Menkes kinky hair syndrome, Cockayne syndrome, Halervorden-Spatz syndrome, lafora disease, Rett syndrome, hepatolenticular degeneration, Lesch-Nyhan syndrome, and Unverricht-Lundborg syndrome), dementia (including, but not limited to Pick's disease, and spinocerebellar ataxia), cancer (e.g. of the CNS and/or brain, including brain metastases resulting from cancer elsewhere in the body), neuropathy, amyloidosis, an ocular disease or disorder, viral or microbial infection, inflammation, ischemia, seizure, behavioral disorders, and a lysosomal storage disease.

A “neurological disorder drug” is a drug or therapeutic agent that treats one or more neurological disorder(s). Neurological disorder drugs envisage in current application include, but are not limited to antibodies, peptides, proteins, natural ligands of one or more CNS target(s), modified versions of natural ligands of one or more CNS target(s), aptamers, inhibitory nucleic acids or antisense oligonucleotides (i.e., small inhibitory RNAs (siRNA), short hairpin RNAs (shRNA) or gapmers), ribozymes, and small molecules, or active fragments of any of the foregoing that either are themselves or specifically recognize and/or act upon (i.e., inhibit, activate, or detect) a CNS antigen or target molecule. A “CNS antigen” or “brain antigen” is an antigen expressed in the CNS, including the brain, which can be targeted with an antibody or small molecule. Non-limiting examples of said CNS antigen or target molecule are amyloid precursor protein or portions thereof, amyloid beta, beta-secretase BACE1, gamma-secretase, Tau, alpha-synuclein, parkin, huntingtin, DR6, presenilin 1, presenilin 2, ApoE, glioma or other CNS cancer markers, and neurotrophins. Non-limiting examples of neurological disorder drugs and disorders they may be used to treat are anti-BACE1 antibodies (e.g. WO2009121948, WO2010146058, WO2012064836) and anti-HER2 antibody (e.g. trastuzumab) (e.g. WO2003087131).

In another aspect of the application, a method of treating a subject is provided, said method comprising the step of administering to said patient the shuttle or (pharmaceutical) composition described above having a koff for human TfR of less than 5×10⁻²/s, or less than 4×10⁻²/s, 3.5×10⁻², 3×10⁻², 2.9×10⁻², 2.8×10⁻², 2.7×10⁻², 2.6×10⁻², 2.5×10⁻², 2.4×10⁻², 2.3×10⁻², 2.2×10⁻², 2.15×10⁻², 2.1×10⁻², 2×10⁻², 1.9×10⁻², 1.8×10⁻², 1.7×10⁻², 1.6×10⁻², 1.5×10⁻²/s, 1.4×10⁻², 1.3×10⁻², 1.2×10⁻², 1.1×10⁻², 1×10⁻², 9×10⁻³, or 8×10⁻³/s as determined by BLI or having a koff for human TfR of between 3×10⁻⁴/s and 3×10⁻²/s, or between 5×10⁻⁴/s and 2×10⁻²/s or between 8×10⁻⁴ and 1×10⁻²/s, between 9×10⁻⁴ and 9×10⁻³/s or between 1×10⁻³ and 8×10⁻³/s as determined by BLI comprising a neurological disorder drug, wherein the subject is suffering from a neurological disorder. Also a method of in vivo medical imaging a body area or tissue of a subject is provided, more particularly a brain region, said method comprises administering to the subject an effective amount of any of the blood brain barrier shuttles herein disclosed comprising an imaging compound, and detecting the imaging compound in body areas of said subject. The method further comprises collecting one or more images of the subject and displaying the one or more images of the subject. The images may be taken over a period of time, including multiple images over a period of time. The collecting and displaying of said images can be done by a commercially available scanner and the accompanying computer hardware and software. For example PET and SPECT scanners may be used. Said imaging compound can be any compound that allows efficient in vivo medical imaging. A non-limiting example is a radionuclide, e.g. Technetium (99mTC) or Lutetium-177. Also provided is a method of transporting the composition or shuttle described herein from the peripheral blood stream in a subject to the CSF, more particularly from the basolateral side of the CPE cells to the apical side, said method comprising the step of administering to said subject any one of the shuttles or (pharmaceutical) compositions described herein.

In one embodiment of above methods, said composition or shuttle is administered to said patient using a route selected from the list consisting of oral administration, nasal administration, intravenous administration, intramuscular administration, subcutaneous administration, transdermal administration, intradermal administration, topical administration and enteral administration. In one embodiment said composition is not administered intracerebrally or intracerebroventricularly or epidurally or not through any alternative direct administration to the brain.

### Anti-Cancer Approaches: TfR-Targeting Anti-Cancer Therapeutics

Surgery, chemotherapy and radiotherapy have long been considered the best options for cancer treatment. However, these therapies are an indiscriminate warfare, coinciding with damaging side effects and failing to protect against recurring cancer cells (Lecocq, 2019). The identification of molecular accelerators of cancer cells, such as HER2, led to the development of molecularly targeted treatments, designed to bind and override faulty molecules in cancer cells (Lecocq, 2019). Modern cancer therapy and diagnostic is focused on targeted and thus specific delivery of high doses of chemotherapeutic drugs or diagnostic agents to tumor sites while sparing normal tissue and thus overcoming the drug's high systemic toxicity. Targeted therapies in the clinic requires high affinity, tumor-specific agents and effective targeting vehicles to deliver therapeutics to the tumor site (Xing, 2018).

As described in Ying Shen et al (2018) Am. J. Cancer Res. 8(6): 916-931 TfR is an attractive anti-cancer target.

Antitumor pro-drugs linked to anti-TfR binding agents as those described in current application can be absorbed within TfR expressing tumor cells based on the molecular ‘Trojan horses’ principle. Because TfR quantitatively recycles between the cell surface and intracellular compartments, the TfR-mediated endocytosis machinery can be used as a portal of entry to deliver large payload of anti-cancer therapeutics (Kalim et al 2017 Drug Des Devel Ther 11).

In another aspect, any of the TfR binding agents of current application are provided for use in in vivo medical imaging or for use to treat cancer, particularly TfR expressing cancers, even more particularly TfR expression cancers selected from the list consisting of ovary, breast, pleura, lung, cervix, endometrium, colon, kidney, bladder and brain cancer. Current application teaches that for transport to the CNS over the BBB the TfR binding agents of the application should have a dissociation constant koff within a specific range. However, for binding to the TfR in or for binding at the surface of TfR expressing cancer cells, said specific dissociation constant is not an essential feature. Hence, any TfR binding agent herein disclosed is provided of use in cancer diagnostic and treatment approaches, for example by coupling to anti-cancer agents or imaging compounds.

In one embodiment, an TfR binding agent is provided with a KD from 50 nM to 500 nM for human TfR, said binding agent when coupled to a chemical entity improves the uptake of the chemical entity into TfR expressing cancer cells or improves the binding of the chemical entity to the surface of TfR expressing cancer cells. In particular embodiments, the TfR binding agent is one of the TfR binding agents from the application. In a most particular embodiment, the TfR binding agent is one of the VHHs from the application.

VHHs have been studied extensively in the context of targeted cancer therapy and immunotherapy.

VHHs are embraced by different types of strategies in the fight against cancer: (1) dampen oncogenic signals, (2) deliver lethal punch to cancer cells, (3) design cancer vaccines, (4) engage cytolytic cells, and (5) prevent immunosuppressive events (Lecocq, 2019).

VHHs lacking antagonistic traits, yet target cancer cells, have been coupled to other technology platforms to deliver a targeted, lethal punch to cancer cells (Lecocq, 2019). VHHs have been coupled to death inducing ligands (e.g. TRAIL), truncated form of Pseudomonas exotoxin A, various drugs and drug-loaded nanoparticles, photosensitisers (i.e. hitting a photosensitizer with light of a particular wavelength in an oxygenated environment results in formation of ROS), therapeutic radionuclides (i.e. radioactive labels such as Lutetium-177, Iodine-131, Astatine-211, Actinium-225 and Bismuth-213 can be used to release their energy in the proximity of cancer cells, thereby causing irreparable DNA damage), and enzymes for prodrug activation (e.g. β-lactamase to convert prodrug 7-(4-carboxybutanamido) cephalosporin mustard in phenylenediamine mustard) (Lecocq, 2019). Similar as photosensitizers, branched gold nanoparticles kill cancer cells when excited by NIR-light, but by generating heat instead of ROS (Lecocq, 2019). VHH can also bring these toxic moieties close to cancer cells, while minimizing toxic effects to healthy tissues, hence reducing potential adverse effects (Lecocq, 2019).

Several bifunctional molecules have been designed (e.g. anti-EGFR VHH coupled to TRAIL) (Lecocq, 2019). Among the drugs that are frequently used to treat various cancer types are cisplatin and its analogues, carboplatin and oxaliplatin as well as doxorubicin, RTK inhibitors and death effector molecules. As these drugs lack selectivity, VHHs have been used to target them to cancer cells (Lecocq, 2019).

In various embodiments, a pharmaceutical composition is provided comprising any of the TfR binding agents of current application coupled to a chemotherapeutic agent for use as a medicament, more particularly for use to treat cancer, even more particularly for use to treat TfR expressing cancers.

Non-limiting examples of said chemotherapeutic agents are alkylating agents such as thiotepa and CYTOXAN cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (e.g., bullatacin and bullatacinone); a camptothecin (including the synthetic analogue topotecan); bryostatin; cally statin; CC-1065 (including its adozelesin, carzelesin and bizelesin synthetic analogues); cryptophycins (e.g., cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as minoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; def of amine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL paclitaxel (Bristol-Myers Squibb Oncology, Princeton, N.J.), ABRAXANE Cremophor-free, albumin-engineered nanoparticle formulation of paclitaxel (American Pharmaceutical Partners, Schaumberg, 111.), and TAXOTERE doxetaxel (Rhone-Poulenc Rorer, Antony, France); chloranbucil; GEMZAR gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE. vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb); inhibitors of PKC-α, Raf, H-Ras, EGFR (e.g., erlotinib (Tarceva)) and VEGF-A that reduce cell proliferation and pharmaceutically acceptable salts, acids or derivatives of any of the above. In addition, the methods of treatment can further include the use of photodynamic therapy.

In some embodiments, the TfR binding agents or the pharmaceutical compositions described herein act synergistically when co-administered with another therapeutic agent. In such embodiments, the TfR binding agent and the additional therapeutic agent may be administered at doses that are lower than the doses employed when the agents are used in the context of monotherapy.

### TfR-Targeting for Tumor Imaging

When diagnosing cancer, one would like to know as much as possible about the tumor, such as the presence of targetable tumor antigens and the immune context, to plan and monitor the most effective treatment (Lecocq, 2019). TfR-targeting for non-invasive imaging of TfR positive primary and metastatic tumors allows reliable patient selection for personalized anti-cancer treatment with TfR-targeting therapeutics and permits whole-body monitoring of the TfR expression status of tumors throughout treatment (Cheung et al 2016 Oncotarget 7). Imaging techniques based on TfR specific agents also assist surgeons in performing better resections in patients with TfR-expressing tumors (Scaranti, 2020).

One approach is the use of labeled TfR binding agents.

VHH-based imaging has been extensively studied to detect cancer cells in preclinical studies (e.g. antigens CEA, EGFR, HER2, PSMA, CD20, CD38) (Lecocq, 2019). For clinical purposes, the most advanced VHH-based imaging agent is 68Ga-coupled anti-HER2 nanobody 2Rs15d for PET imaging of BC patients (Lecocq, 2019). The first clinical trial in 2016 revealed that HER2 in primary tumors and local or distant metastases could be detected and imaged as soon as 60 minutes post-injection without adverse effects, such as renal toxicity and tracer-induced antibodies and was highly specific (Lecocq, 2019). Moreover, background uptake was very low with the exception of signals observed in the kidneys, intestines and liver (Lecocq, 2019). Recently, a phase II clinical trial evaluating the potential of 68Ga-NOTA-2Rs15d to detect brain metastasis has been initiated (NCT03924466) (Lecocq, 2019). Implementation of VHH-based imaging of cancer markers can be a guide for therapy selection, in particular as targeted therapies have been developed for many of these cancer markers, some of which are based on the use of VHH (e.g. anti-HER2 VHH for targeted therapy) (Lecocq, 2019). Moreover, VHH-based probes have been developed to image the expression of immune checkpoints (Lecocq, 2019).

For noninvasive imaging, VHHs need to be labeled with an imaging probe that can consist of a (1) radioisotope, (2) fluorescent dye, (3) microbubble or (4) a chemical like gadolinium, allowing imaging via technologies such as single-photon emission computed tomography (SPECT), positron emission tomography (PET), optical imaging (01), ultrasound (US) and MRI (Lecocq, 2019). The majority of VHH-mediated imaging studies use SPECT and PET, because these radioisotope-based techniques have a high sensitivity, resolution and offer quantitative information (Lecocq, 2019). In preclinical studies, VHHs often contain a genetically inserted C-terminal hexahistidine tag for purification purposes, which can be complexed with 99mTc (CO3), a γ-emitting radionuclide that is easily detectable using SPECT (Lecocq, 2019). For PET, which is clinically more relevant, VHHs are labeled with positron-emitting radionucles (18F (half-life 68 min), 64Cu, 68Ga (half-life 110 min) and 89Zr) (Lecocq, 2019). The half-lifes match the biological half-life of VHHs when injected i.v. (Lecocq, 2019). Site-specific labelling is desired to obtain homogenous and consistent tracers (e.g. transpeptidase sortase A-mediated ligation, catalizing formation of peptide bond between C-terminally expressed LPXTG peptide motif of the VHH and the N-terminal oligo-glycine motif on the label) (Lecocq, 2019). An alternative to radiolabelling of VHHs is the use of fluorescent dyes that can be combined with optical imaging (01). For in vivo imaging, NIR emitting fluorophores (e.g. IRDye-680RD or -800CW, Cy5 and AlexaFluor 680) are the label of choice, as these provide strong contrast and resolution combined with signal detection in depths ranging from several hundred um to one cm (Lecocq, 2019). Advantages of 01 are its flexibility, simplicity and cost-effective character, as in contrast to radioisotope-mediated imaging, it does not require dedicated facilities (Lecocq, 2019). OI is often used to study surface lesions during surgical or endoscopic procedures, as 01 dyes have limited tissue penetrating capacity compared to radioisotope-based imaging (Lecocq, 2019). US can be used as an alternative to radiolabelled VHHs while retaining the ability for high-resolution images (Lecocq, 2019). US requires conjugation of VHHs to US contrast agents, microbubbles or nanobubbles that allow the molecular characterization of the vascular wall (after i.v. administration) (Lecocq, 2019). In MRI imaging, VHH-coated superparamagnetic nanoparticles allow antigen detection in xenografted tumors (Lecocq, 2019).

### TfR Binding VHHs for Use in Theranostics

A growing modality in precision oncology is the development of theranostics, as this enables patient selection, treatment and monitoring (Lecocq, 2019). In this approach, labelled compounds and an imaging technology are used to diagnose patients and select the best treatment option, whereas for therapy, related compounds are used to target cancer cells or the tumor stroma (Lecocq, 2019). In this context, VHHs and VHH-directed therapeutics have gained interest (Lecocq, 2019). This interest stems from their high antigen specificity, small size, ease of labelling and engineering, allowing specific imaging and design of therapies targeting antigens on tumor cells, immune cells as well as proteins in the TME (Lecocq, 2019).

In oncology there is a growing interest in targeted radionuclide therapy (TRNT) that selectively delivers radioactivity and kills malignant cells, while minimizing the harm to healthy cells (Ersahin et al., 2011). Due to the widespread availability of therapeutic radionuclides, this therapy strategy is gaining more attention (Tomblyn et al., 2012). Radioimmunotherapy (RIT) is a TRNT strategy that employs radiolabeled monoclonal antibodies (mAbs) that interact with tumor-associated proteins that are expressed on the cancer cell surface and thus readily accessible by these circulating agents. For the treatment of B cell Non-Hodgkin's lymphoma (NHL) RIT consists of the radiolabeled anti-CD20 mAbs ⁹⁰Y-ibritumomab tiuxetan (Zevalin) and ¹³¹I-tositumomab (Bexxar). Zevalin is now FDA approved as a late-stage add-on to the unlabeled anti-CD20 mAb Rituximab for the treatment of relapse and refractory NHL. Due to the high radiosensitivity of lymphomas only a relatively low absorbed dose is required to obtain an objective response. Although recent clinical trials have shown beneficial effect of the combination of Rituximab and Zevalin versus Rituximab alone (Tomblyn et al., 2012), Zevalin has only been approved for late-stage disease (patients with disease recurrence or non-responders to chemotherapy and immunotherapy with Rituximab).

VHHs have superior characteristics compared to classical mAbs and their derived fragments for in vivo cell targeting (De Vos et al., 2013). In terms of molecular imaging of cancer, VHHs have been directed to a variety of membrane-bound cancer cell biomarkers, such as CEA, EGFR, HER2, and PSMA (D'Huyvetter et al., 2014). Because of their exceptional specificity of targeting, and the fact that they show to be functional after labeling with radionuclides, VHHs became valuable vehicles for nuclear imaging and TRNT (D'Huyvetter et al., 2014).

Diagnostic tests like IHC are current practice but are unable to portray whole tumor expression levels and this is even worse for metastatic lesions (Lecocq, 2019). Indeed, this could explain the failure to accurately predict outcome responses in all patients. Whole body, non-invasive imaging modalities such as PET, SPECT, MRI and OI, using VHH-based tracers, could fulfil these shortcomings and could be implemented repetitively without the need of collecting invasive biopsies (Lecocq, 2019). Intriguingly, many of the described VHHs hold the potential to be used as molecular imaging probes as well as therapeutic agents. The term “theranostic” was initially put forward to describe the development of diagnostic tests alongside the application of a therapy targeted towards a specific molecular feature (Lecocq, 2019). Currently, the term theranostics is used in a much stricter sense and rather refers to agents that are identical or closely related and that harbour the potential to be used both for diagnostic as well as for therapeutic purposes (Lecocq, 2019). VHHs targeting cancer-specific membrane proteins (e.g. HER2) have been evaluated for both imaging and therapeutic applications (Lecocq, 2019). The clearest example of VHH theranostics is where both diagnostic tracers and therapeutic compounds are radiolabelled, in a TRT approach (Lecocq, 2019). The radiolabel can be different (Gallium-68 or Fluor-18 for PET imaging and Actinium-225 for α-TRT), but sometimes the radiolabel is the same, such as Iodine-131 labelled VHHs that are first used at low doses in SPECT imaging for diagnosis and dose estimations, and then at higher doses for TRT (Lecocq, 2019). Of importance, diagnostic and therapeutic VHH-radiopharmaceuticals have similar pharmacokinetics and biodistribution profile (Lecocq, 2019).

In one embodiment, any of the TfR binding agent of the application coupled to a radionuclide is provided. In an embodiment, the TfR binding agent is coupled or fused to the radionuclide either directly or through a coupling agent and/or a linker and/or a tag. In a specific embodiment, the TfR binding agent is fused to the radionuclide via a His-tag. Methods used for radiolabelling the TfR binding agent are conventional methods and are known to persons skilled in the art. Any available method and chemistry may be used for association or conjugation of the radionuclide to the TfR binding agent. As an example, tricarbonyl chemistry may be used for radiolabeling (Xavier et al. 2012). In certain embodiments, the TfR binding agent is coupled to a radionuclide that is damaging or otherwise cytotoxic to cells and the TfR binding agent targets the radionuclide to TfR expressing cells, preferentially to cancerous cell. The radiolabelled TfR binding agent is used, for example—but not limited to—to target the damaging radionuclide to cancer tissue to preferentially damage or kill cancer cells.

According to particular embodiments, any of the TfR binding agent described herein is useful for targeted radionuclide therapy. “Targeted radionuclide therapy”, as used herein, refers to the targeted delivery of a radionuclide to a disease site and the subsequent damage of the targeted cells and adjacent cells (bystander effect). In targeted radio-therapy, also referred to as systemic targeted radionuclide therapy (STaRT), the biological effect is obtained by energy absorbed from the radiation emitted by the radionuclide. Non-limiting exemplary radionuclides are Iodine-131, Astatine-211, Bismuth-213, Lutetium-177 or Yttrium-86. Exemplary radionuclides that can be used to damage cells, such as cancer cells, are high energy emitters. For example, a high energy radionuclide is selected and targeted to cancer cells. The high energy radionuclide preferably acts over a short range so that the cytotoxic effects are localized to the targeted cells. In this way, radio-therapy is delivered in a more localized fashion to decrease damage to non-cancerous cells.

The present invention also pertains to the use of the TfR binding agents described herein for disease diagnosis and/or prognosis and/or treatment prediction in a subject. As non-limiting example, a subject having cancer or prone to it can be determined based on the expression levels, patterns, or profile of TfR in a test sample from the subject compared to a predetermined standard or standard level in a corresponding non-cancerous sample. In other words, TfR polypeptides can be used as markers to indicate the presence or absence of cancer or the risk of having cancer, as well as to assess the prognosis of the cancer and for prediction of the most suitable therapy.

In a further related aspect, the disclosure contemplates a pharmaceutical composition comprising any of the TfR binding agent as described herein, in association with a pharmaceutically acceptable carrier. Therefore, the TfR binding agent alone or coupled to chemical agent (see above) may be formulated in a physiologically or pharmaceutically acceptable carrier suitable for in vivo administration. In certain embodiments, such compositions are suitable for oral, intravenous or intraperitoneal administration. In other embodiments, such compositions are suitable for local administration directly to the site of a tumor. In certain embodiments, such compositions are suitable for subcutaneous administration.

### Methods of Treatment

In another aspect of the application, a method of treating a subject is provided, said method comprising the step of administering to said patient a composition comprising one of the TfR binding agents of current application coupled to a cancer drug, wherein the subject is suffering from cancer.

Also a method of binding an TfR binding agent to a cancer tissue is provided, more particularly a TfR-expressing cancer tissue, comprising the step of administering a composition comprising one of the TfR binding agents of current application to the cancer tissue. Also a method of directing a compound to a cancer cell or tissue, more particularly a TfR-expressing cancer cell or tissue, comprising the step of administering a composition comprising the compound coupled to any of the TfR binding agents of current application to a cancer cell or tissue. In one embodiment, said cancer cell or tissue is present in a mammal, more particularly a human. In another embodiment, said cancer cell or tissue is an in vitro cancer cell or tissue. In yet another embodiment, said compound is any of the cytotoxic or chemotherapeutic compounds or any imaging compound herein described.

In one embodiment, a method of administering or transferring or directing a cancer drug or an imaging compound to a TfR expressing cancer cell is provided, comprising administering a composition to a subject comprising any of the herein disclosed TfR antibodies coupled to a cancer drug or an imaging compound.

In one embodiment of above methods, said composition is administered to said patient using a route selected from the list consisting of oral administration, nasal administration, intravenous administration, intramuscular administration, subcutaneous administration, transdermal administration, intradermal administration, topical administration and enteral administration. In one embodiment said composition is not administered intracerebrally or intracerebroventricularly or epidurally or not through any alternative direct administration to the brain.

### In Vivo Medical Imaging Method

In another aspect, the disclosure provides an in vivo medical imaging method. The method comprises administering to a subject, such as a human or non-human subject, an effective amount of the labelled TfR binding agent as described herein. The effective amount is the amount sufficient to label the desired cells and tissues so that the labelled structures are detectable over the period of time of the analysis. The method further comprises collecting one or more images of the subject and displaying the one or more images of the subject. The images may be taken over a period of time, including multiple images over a period of time. The collecting and displaying of said images are done by a commercially available scanner and the accompanying computer hardware and software. For example PET and SPECT scanners may be used. Said imaging compound can be any compound that allows efficient in vivo medical imaging. A non-limiting example is a radionuclide, e.g. Technetium (99mTC) or Lutetium-177. Moreover, to further improve the usefulness of the images generated, CT, X-ray or MRI may be simultaneously or consecutively used to provide additional information, such as depiction of structural features of the subject. For example, dual PET/CT scanners can be used to collect the relevant data, and display images that overlay the data obtained from the two modalities. By way of example, when selecting a radionuclide for in vivo imaging, a gamma or positron emitting radionuclide or a radionuclide that decays by electron transfer may be preferred. Emissions can then be readily detected using, for example, positron emission tomography (PET) or single photon emission computed tomography (SPECT). Generally, it is desirable that the half-life of the radionuclide is long enough to be made and used in testing, but not so long that radioactivity lingers in the patient for a considerable period of time after the test has been performed. Moreover, the amount of radioactivity used to label can be modulated so that the minimum amount of total radiation is used to achieve the desired effect.

It is to be understood that although particular embodiments, specific configurations as well as materials and/or molecules, have been discussed herein for cells and methods according to the present invention, various changes or modifications in form and detail may be made without departing from the scope and spirit of this invention. The following examples are provided to better illustrate particular embodiments, and they should not be considered limiting the application. The application is limited only by the claims.

### EXAMPLES

### Example 1. Identification of Human/Cynomolgus TfR Binders

Currently described TfR affinity binders able to cross the blood-brain barrier all bind TfR apical domain. However, previous experience from our lab and others indicate that acquiring human and cynomolgus TfR binders is challenging. Despite the fact that the apical domains of human and cynomolgus TfR have a 95% homology and only differ in 9 amino acids, it has been shown that this can already yield in poor or no binding to the cynomolgus TfR sequence (Kariolis et al 2020 Sci Transl Med 12). In addition, we have observed that two residues in the cynomolgus sequence that differ from the human sequence, give rise to a glycosylation site that is absent in the human TfR, which could also influence the binding of biologicals to TfR. Therefore, in this study the immunization strategy was reconsidered. In order to obtain BBB-crossing VHHs that are both human and cynomolgus cross-reactive, camelids were immunized with DNA encoding for alpaca TfR with the sequence encoding for the apical domain replaced by the cynomolgus apical domain sequence.

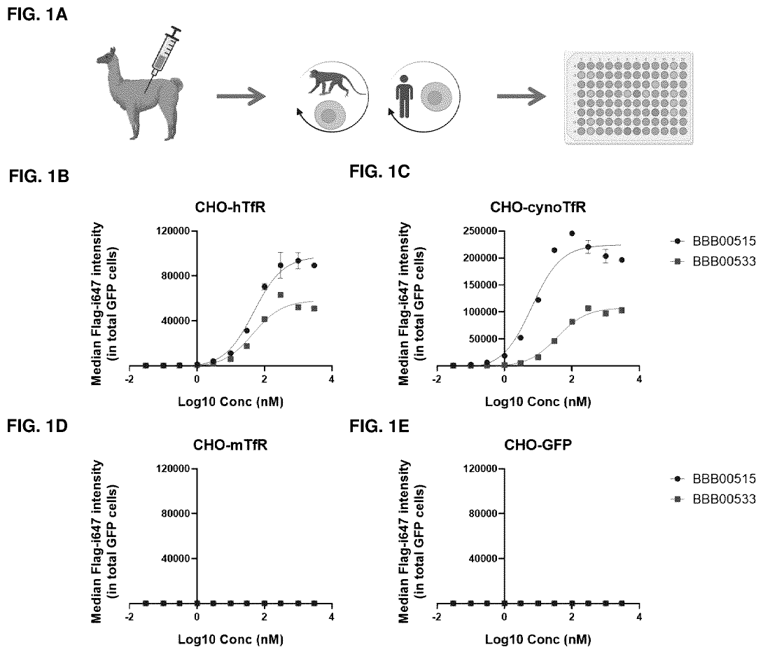

Phage libraries generated after 4 biweekly immunizations of two different camelids, underwent two rounds of pannings on CHO cell lines overexpressing either cynomolgus TfR in the first round or human TfR in the second round. Output libraries were screened with flow cytometry to find binders to human TfR overexpressing cells (FIG. 1A). With this strategy we enriched for human/cynomolgus binders that were able to bind TfR in living cells. Two lead VHHs, BBB00515 and BBB00533 bound both human and cynomolgus TfR overexpressing cells whereas they did not bind mouse TfR overexpressing cells or a control cell line only expressing GFP (FIG. 1B-E). Binding kinetics to recombinant human and cynomolgus TfR were further characterized with Surface Plasmon Resonance (SPR). Both BBB00515 and BBB00533 bound immobilized recombinant cynomolgus TfR with a similar estimated affinity constant (KD=63.00±1.20 nM for BBB00515 and KD=103.77±8.14 nM for BBB00533; FIG. 1F). Both bound also to immobilized recombinant human TfR but with a higher KD (KD=1183.67±423.81 nM for BBB00515 and KD=207.00±27.84 nM for BBB00533 (FIG. 1F).

### Example 2. Anti-Human/Cynomolgus TfR Nanobodies Shuttle Anti-BACE1 mAb into the Brain

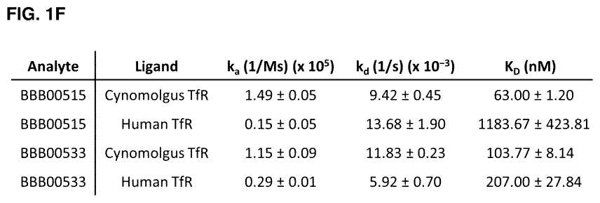

BACE1 inhibition in the brain was the paradigm used to assess the potential of the VHHs to cross the BBB and deliver a biological in the brain. BACE1 is responsible for the β-secretase cleavage on APP (Sinha et al 1999 Nature 402, 537-540). BACE1 inhibiting antibody (Mab 1A11) is able to reduce brain Aβ1-40 levels in vivo but does not cross the BBB (Zhou et al 2011 J Biol Chem 286, 8677-8687). Bispecific antibodies with one intact 1A11 arm and the VHHs on the other arm were engineered and expressed in CHO cells (FIG. 2A). BBB00574 bispecific antibody carries BBB00515 VHH, whereas BBB00578 carries BBB00533 VHH. As expected both bispecific antibodies were still able to bind hTfR in living cells, but not to a negative control cell line (FIG. 2B-C). Binding to BACE1 was confirmed with bilayer interferometry (BLI), in which biotinylated recombinant human BACE1 protein was immobilized at the tip of streptavidin coated biosensors (FIG. 2D-F). Both bispecific antibodies bound human BACE1 with a similar KD of 0.3 nM (FIG. 2F).

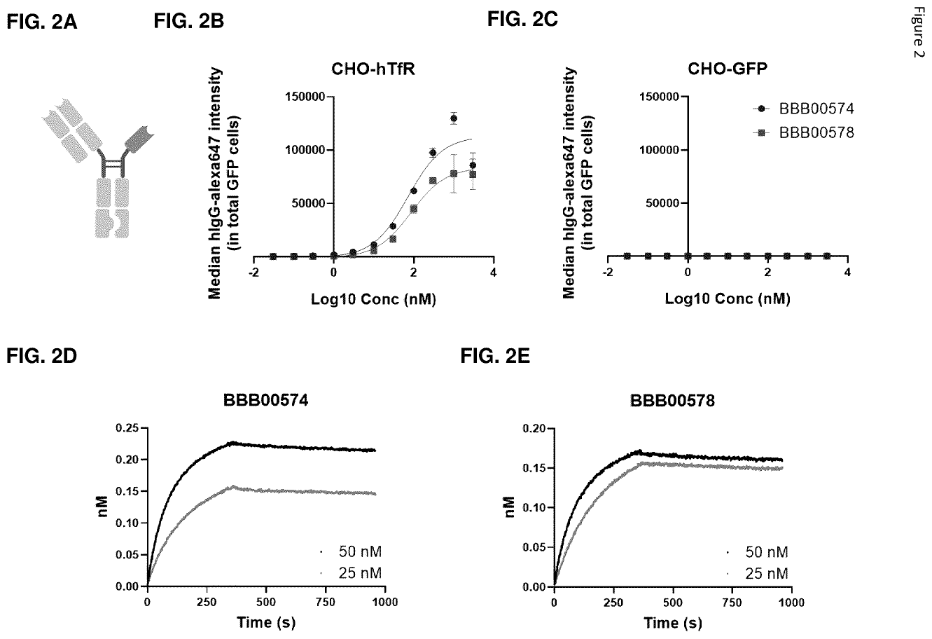

Next, both bispecific antibodies were administered intravenously in a chimeric mouse model in which the mouse TfR apical domain is replaced by the human sequence (hAPI KI mice) (Wouters et al 2022 Fluids Barriers CNS 19, 79). The chosen concentration to inject was 167 nmol/kg, the dose at which no central BACE1 inhibition is observed for mAb 1A11 after peripheral injection (Wouters et al 2022 Fluids Barriers CNS 19, 79; Zhou et al 2011 J Biol Chem 286, 8677-8687; Atwal et al 2011 Sci Transl Med 3). Plasma and brains were harvested 24 hours later and Aβ1-40 levels were quantified with ELISA. Both BBB00574 and BBB00578 bispecific antibodies could lower Aβ1-40 levels in plasma by 60%, as compared to samples of PBS injected mice, but also in the brain by 40%, confirming the ability of both VHHs to carry a biological moiety over the BBB (FIG. 3).

In summary, the above-described results clearly demonstrate that there herein described VHHs not only bind to both human and NHP TfR, but are also able to deliver a biological across the BBB. These VHH have thus the potential to be used in the clinic to increase the brain permeability of a therapeutic and/or diagnostic biological.

### Example 3. Lead Optimization of BBB00515 and BBB00533

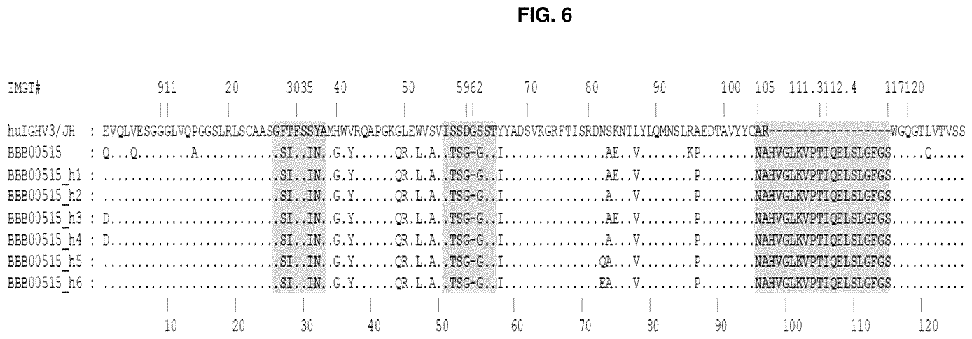

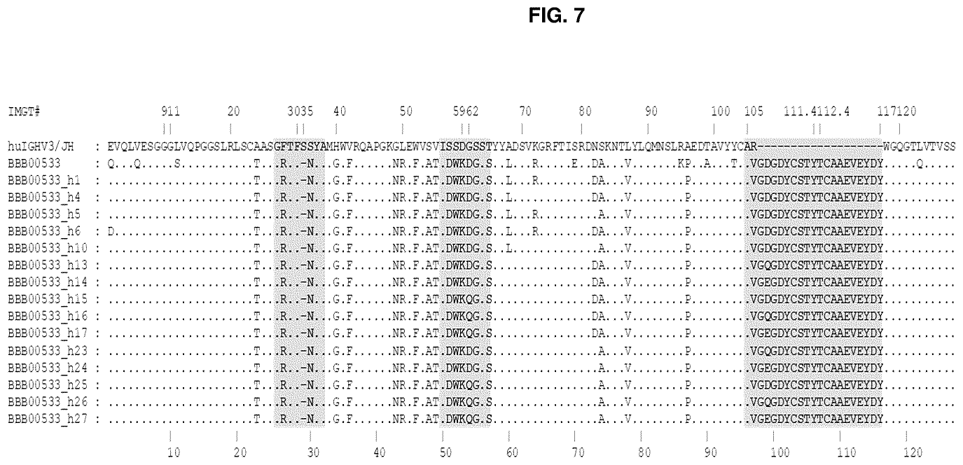

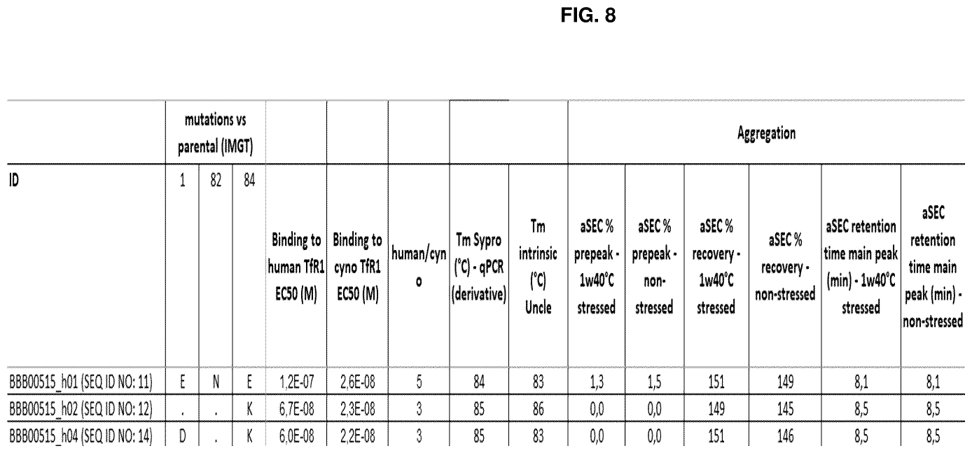

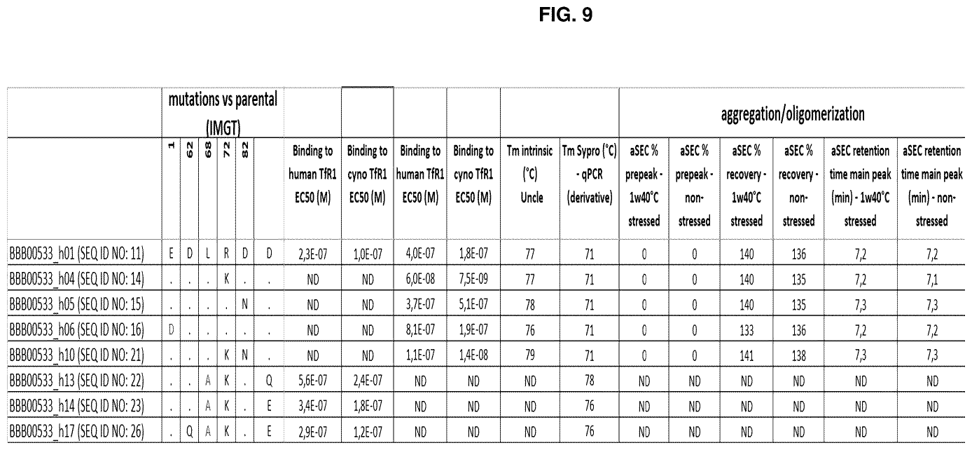

The protein sequences of leads BBB00515 and BBB00533 were modified to improve them in terms of humanization towards human IGHV3 and JH germline consensus sequences, as well as in terms of chemical and biophysical stability, while minimizing the impact on target binding and inhibition. To this end, different variants were generated for each lead (variants of BBB00515 are depicted in SEQ ID NO: 11 to 16, variants of BBB00533 are depicted in SEQ ID NO: 17 to 31). A sequence alignment of the BBB00515 variants is depicted in FIG. 6, a sequence alignment of the BBB00533 variants is depicted in FIG. 7. Residue numbering and CDR delineations were done according to the IMGT nomenclature (Lefranc M P and Lefranc G (2023) Computer-aided antibody design pp 3-59, Springer US). Variants were compared for their capacity to compete for binding on human and cynomolgus monkey TfR1 by means of flow cytometry. Melting (Tm) temperatures of the different variants were also determined. The variants (1 mg/ml in PBS) were also subjected to temperature stress (1 week @40° C.) followed by analytical size exclusion chromatography (aSEC) to assess the oligomerization propensity. In addition, they were subjected to long term temperature stress (1 mg/ml in PBS, 4 weeks @40° C.) and forced oxidative stress (10 mM H₂O₂ for 3 hours @37° C.), followed by detailed peptide mapping mass spectrometry to assess the amino acid stability (see FIGS. 8 and 9 for a summary of the results obtained). Also, the following post-translational modifications were evaluated: deamidation (Asn/Gln), isomerization (Asp), oxidation (Met/Trp), N-terminal cyclization (pyroGlu) and C-terminal truncations. Amino acid residues with poor chemical stability (>5% modification after relevant stress) were replaced by suitable alternatives.

HIS6-only tagged sequence optimization variants of BBB00515 were compared to reference variant BBB00515_h1 (see FIG. 9). Accelerated temperature and oxidative stress experiments with BBB00515_h1 revealed 10% pyroglutamate cyclization of the N-terminal E1 residue. For biologics fusion constructs with BBB00515 in the N-terminal position, the E1D substitution would eliminate this sensitivity without impacting on the binding. The N82 residue displays a minor (6%) deamidation sensitivity. The only other explored mutation (E84K) was well tolerated in terms of binding and biophysical characteristics.

HIS6-only tagged sequence optimization variants of BBB00533 were compared to reference variant BBB00533_h1 (see FIG. 8). Accelerated temperature and oxidative stress experiments with BBB00533_h1 revealed 10% pyroglutamate cyclization of the N-terminal E1 residue. For biologics fusion constructs with BBB00533 in the N-terminal position, the E1D mutation would eliminate this sensitivity without impact on the binding. Despite the presence of two CDR-based potential aspartate isomerization sites (D62 and D108), accelerated stress experiments revealed no major modifications in these two positions. N49Q and L68A substitutions had a strong negative impact, whereas the D82N only had a minor negative impact on the binding properties. The R72K substitution on the other hand substantially improved binding properties. The L68A substitution improved the thermal stability by 7° C.

### Example 4. Comparison with VHH Sequences Disclosed in WO2020144233

The BBB00515 and BBB00533 sequence optimized leads were compared to exemplary humanized variants of TfR1 VHHs (herein depicted as SEQ ID NO: 33, 34, 35 and 36) which sequences are also disclosed in patent application WO2020144233 (Vect-Horus). From a therapeutic biologics developability perspective, the Vect-Horus VHHs display a number of severe liabilities.

First, a good chemical stability profile (minimizing drug product heterogeneity from stress-induced post-translational modifications) is a crucial developability feature. Sequence analysis reveals that the Vect-Horus VHHs have a methionine residue in their CDR3 sequences (M106). CDR-based methionine residues are known to be liable to extensive oxidation. In contrast, BBB00515 and 533 variants have no CDR-based methionine residues and no major stress-induced post-translational modifications).

In a next step we compared the thermal stability (resistance to temperature-induced protein unfolding) which is another crucial aspect of developability for biologics. We found that the Vect-Horus VHH sequences (depicted herein as SEQ ID NO: 33, 34, 35 and 36) have a substantially poorer thermal stability profile (with Tm values ranging from 64 to 70° C.) in comparison to the different BBB00515—(84 to 85° C.) and 533 (71 to 77° C.) variants.

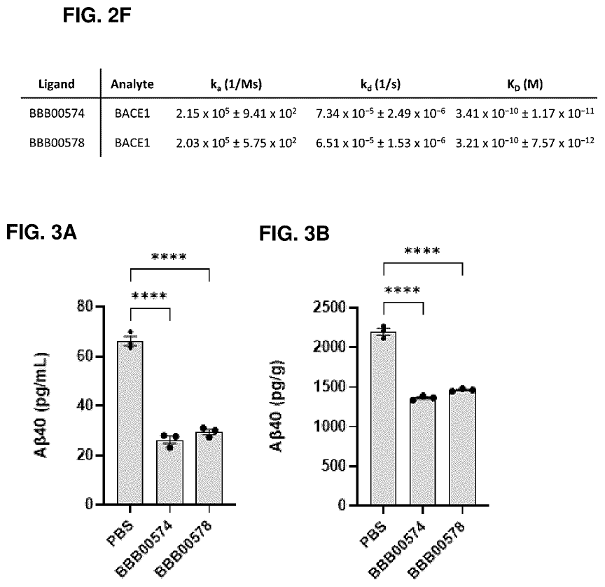

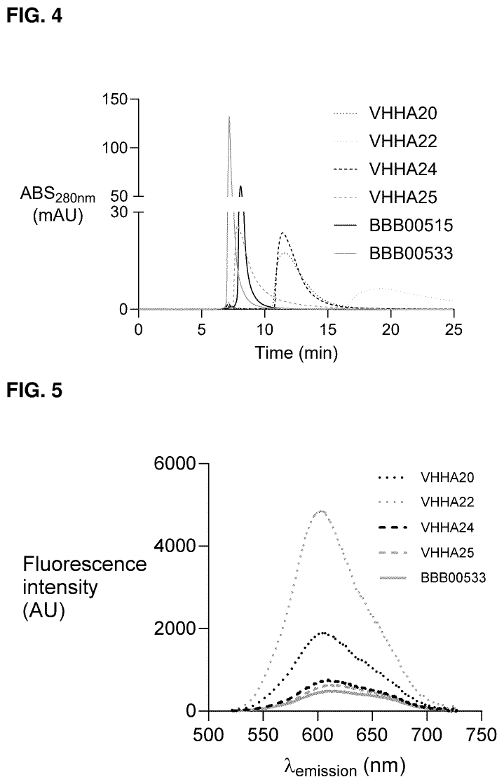

In addition, the absence of stickiness, a good solubility, and low oligomerization and low aggregation propensities are also crucial for developability. In contrast to the sharp and well defined peaks of BBB00515 and 533, the 4 Vect-Horus humanized variants (VHHA25 (SEQ ID NO: 33), VHHA24 (SEQ ID NO: 34), VHHA22 (SEQ ID NO: 35) and VHHA20 (SEQ ID NO: 36) display aSEC profiles indicative of stickiness (substantial column interaction leading to longer elution times), product heterogeneity (broad and a-symmetric peak shapes) and oligomerization (pre-peaks) (see FIG. 4)). Compared to BBB00533, the Vect-Horus humanized variants have much a more pronounced Sypro Orange fluorescence spectrum at room temperature (see FIG. 5), indicative of a higher surface-exposed hydrophobicity in the folded state of the VHHs.

### Example 5—Engineered Variants for Improved Blood-Brain Barrier Shuttling

The side chain of histidine has a pKa of 6.04. Above this pH, histidine has a neutral pH and below this pH it will be positively charged. When present or engineered in antibody CDRs, this may lead to pH dependent binding of such antibodies. Multiple examples are available in the art where antibodies show pH dependent binding (see Maeda K et al (2002) J. Control Release 82(1): 71; Klaus T and Deshmukh S (2021) J. Biomed. Sci. 28(1):11 and Schröter et al (2015) Mabs 7(1): 138). In the context of blood-brain barrier shuttling, this might be of particular importance as some blood-brain barrier shuttling receptors shuttle this barrier through the endosomes (including transferrin receptor). During this process, the endosomes acidify which can lead to release of antibody shuttles binding to such receptors and more efficient blood brain barrier crossing, as shown by multiple research groups (Yogi A et al (2022) Pharmaceutics 14(7):1452; Edavettal S et al (2022) MED. 3(12):860 and Esparza T J et al (2023) Fluids and Barriers of the CNS 20, 1:64).

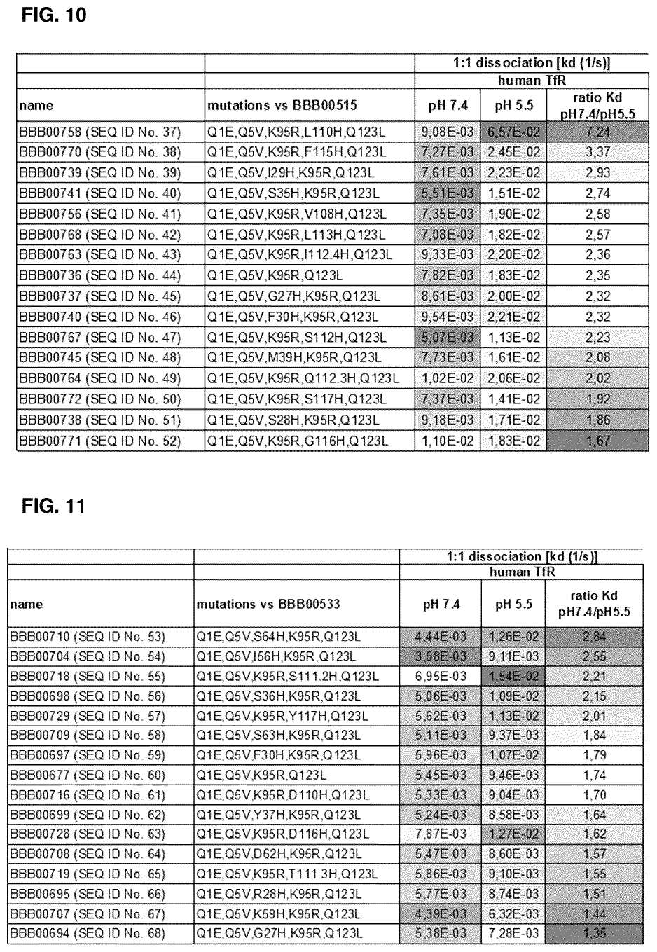

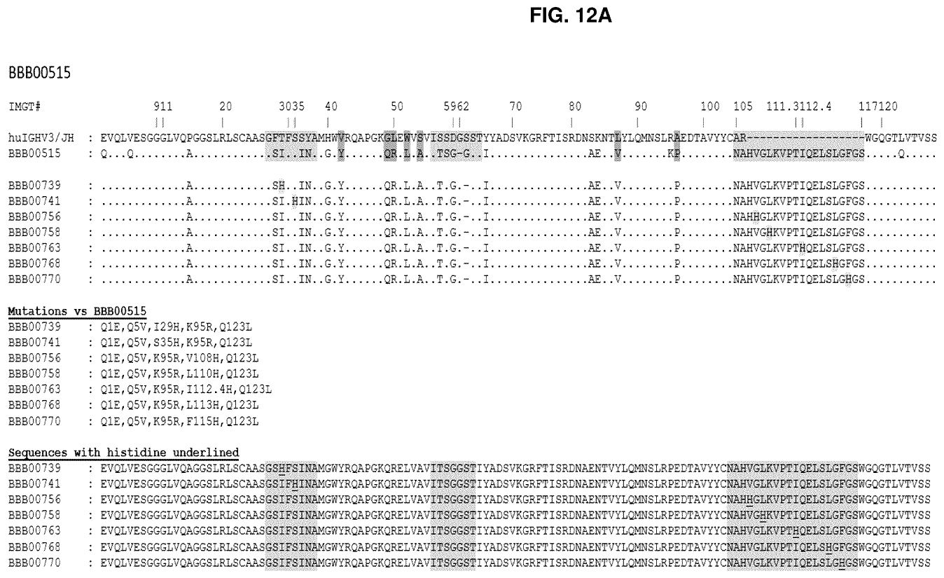

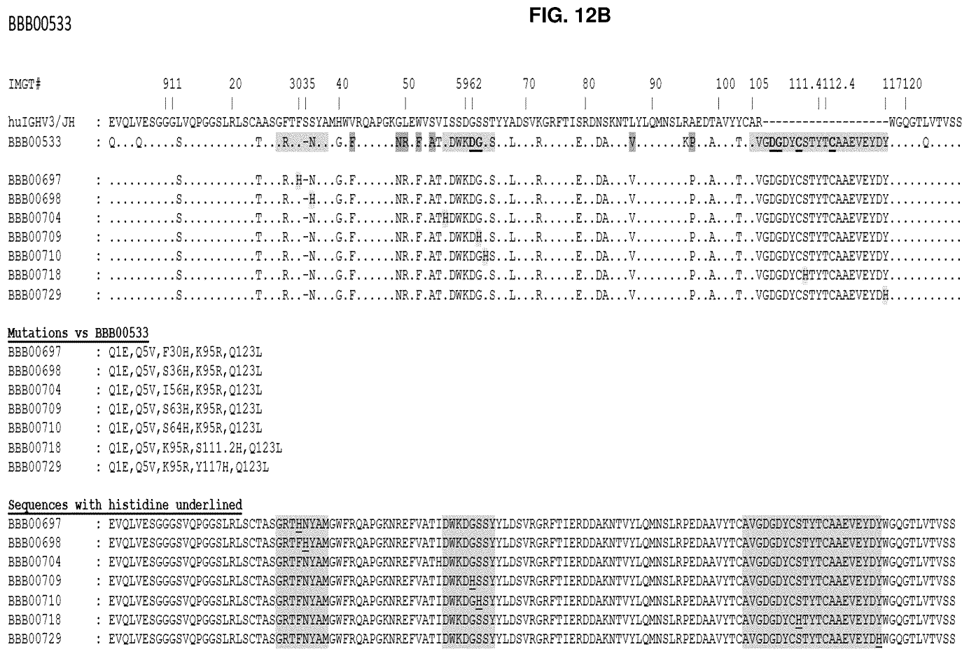

For both BBB00515 and BBB00533, individual mutants were generated where each CDR residue (AbM definition) was replaced by a histidine residue and their pH dependent binding was assessed. In brief, DNA was synthesized by Genscript (Piscataway, New Jersey, United States) and cloned into an E. coli compatible expression plasmid according to standard methods. E. coli TG1 bacteria were transformed with those plasmids and expressions induced. Crude extracts were prepared and analyzed for binding by flow cytometry on human TfR overexpressing cells. The top 50% of clones binding those cells at neutral pH were selected to be re-expressed and purified by immobilized metal affinity chromatography. Next, off rates of these selected variants were determined on recombinant human TfR by surface plasmon resonance (Biacore, Marlborough, Massachusetts, United States). Biotinylated human TfR was captured on a streptavidin-coated SA sensor chip (Cytiva). Increasing concentrations of nanobodies were sequentially injected in a single cycle at a flow rate of 30 μL/min. The dissociation was monitored for around 20 min. A reference flow was used as a control for non-specific binding and refractive index changes. Several buffer blanks were used for double referencing. Off rates were derived after fitting the experimental data to the 1:1 binding model with the Biacore Evaluation Software. Results can be found in FIGS. 10 and 11. FIGS. 12A-12B shows details of the histidine mutants for BBB00515 and BBB00533.

Off rate were compared to the off rates of BBB00736 and BBB00677, variants of respectively BBB00515 and BBB00533 (those variants include some mutations in the frameworks which do not affect the affinities of the VHHs). For BBB00515, following variants were identified with an improved off rate ratio pH7.4/pH5.5 compared to BBB00736: BBB00697, BBB00698, BBB00704, BBB00709, BBB00710, BBB00718 and BBB00729 with ratio's ranging from 1.79 to 2.84. For BBB00533, following variants were identified with an improved off rate ratio pH7.4/pH5.5 compared to BBB00677: BBB00739, BBB00741, BBB00756, BBB00758, BBB00763, BBB00768 and BBB00770 with ratio's ranging from 2.63 to 7.24.

These variants with an improved ratio could potentially have a better BBB crossing efficiency compared to variants with the respective wild-type amino acids at those positions. As shown by Esparza et al. (2023) Fluids and barriers of the CNS 20, no. 1: 64), combining two or more mutations in a single VHH could lead to an even improved off-rate and hence even more improved BBB crossing.

### Experimental Procedures

### Materials and Methods

### Animals

All animal experiments were conducted according to protocols approved by the local Ethical Committee of Laboratory Animals of the KU Leuven (governmental license LA1210579, ECD project number P213/2020) following governmental and EU guidelines. Humanized Tfrc mice, which express a chimeric mouse TfR with the human apical domain under the endogenous promoter, were used for this study (Wouters et al 2022 Fluid Barriers CNS 17, 62).

### Immunization and Nanobody Library Preparation

Targeted VHH libraries were obtained in collaboration with the VIB Nanobody Core (VIB, Belgium). Three alpacas were subjected to four bi-weekly DNA immunizations using recombinant pVAX1 plasmid DNA (Thermo Fisher Scientific) encoding for a chimeric alpaca TfR with the cynomolgus apical domain (synthesized at Twist Biosciences). DNA solutions were injected intradermally at multiple sites at front and back limbs near the draining lymph nodes followed by electroporation. On day 4 and 8 after the last immunization, blood samples were collected, pooled and total RNA from peripheral blood lymphocytes was isolated to recover the nanobody encoding genes. The phagemid library was prepared as previously prescribed (Pardon et al 2014 Nat Protoc 9, 674-693). Briefly, total RNA was used as template for first strand cDNA synthesis with oligodT primer. This cDNA was used to amplify the nanobody-encoding open reading frames by PCR, digested with PstI and NotI, and cloned into a phagemid vector (pBDS001, a modified pMECS vector with an insertion of 3×Flag/6×His tag at the C-terminus of the nanobody insertion site). Electro-competent E. coli TG1 cells were transformed to obtain the nanobody libraries.

### Cell Line Generation

The Flp-In™-CHO™ system (Thermo Fisher Scientific) was used to generate stable CHO cell lines overexpressing cynomolgus or human TfR. DNA encoding for the cynomolgus or the human TfR followed by an HA tag and IRES-GFP was synthesized and subcloned by Twist Bioscience into the pcDNA™5/FRT mammalian expression vector (Thermo Fisher Scientific). Flp-In™-CHO™ cells were maintained with Gibco™ Ham's F-12 Nutrient Mix medium supplemented with GlutaMAX™ (Thermo Fisher Scientific) and 10% FBS and 100 μg/mL Zeocin™ selection antibiotic (Invivogen) until the day of transfection. Cells were transfected with TransIT-PRO® Transfection kit (Mirus) and maintained in Gibco™ Ham's F-12 Nutrient Mix medium supplemented with GlutaMAX™ (Thermo Fisher Scientific) and 10% FBS and Hygromycin B Gold (Invivogen) to select for stable transfectants. Stable transfectants were then amplified and frozen with 10% DMSO for further use.

### VHH Selection, Expression and Purification

VHH-displaying M13 phage libraries were prepared according to standard protocols (Pardon et al 2014 Nat Protoc 9, 674-693), and selected twice on TfR overexpressing cells. Briefly, 6×10¹¹ cfu of phages were blocked with PBS/10% FBS and incubated for an hour with a 5 million cell aliquot containing either CHO-cynomolgus TfR overexpressing cells in the first selection round, or CHO-human TfR overexpressing cells in the second selection round. Non-binding phages were discarded with 5 consecutive washing steps with PBS/10% FBS, whereas bound phages were eluted by trypsinization. Second selection round output phage library was subcloned into an expression vector (pBDS119, a modified pHEN6 vector with an OmpA signal peptide and a C-terminal 3×Flag/6×His tag) and transformed in E. coli TG1 cells. Single clones were picked and sequenced and clustered according to sequence homology. In addition, small scale expression of sequenced clones was performed and periplasmic extracts were prepared as previously described (Pardon et al 2014 Nat Protoc 9, 674-693) to screen for direct binding to CHO-human TfR overexpressing cells. VHH leads were expressed and purified by Immobilized Metal Affinity Chromatography (IMAC) according to the protocol by Pardon et al. (Nat Protoc 9, 674-693).

### Flow Cytometry-Based Binders Screening and Validation

Periplasmic extracts diluted 1:10 in PBS 2% FBS, or a dilution range of different VHH or bispecific antibody concentrations prepared in PBS 2% FBS, were incubated with 0.1 million CHO cells overexpressing either the human, cynomolgus or mouse TfR for 30 min at 4° C. As control for background binding, periplasmic extracts, VHHs or bispecific antibodies were also incubated with 0.1 million CHO cells overexpressing GFP. Binding of VHHs was next resolved by a second step 30 min incubation at 4° C. with an anti-FLAG-iFluor647 antibody (A01811, Genscript) diluted 1:500 for screening and 1:250 for validation assays, or with anti-human IgG Fc-Alexa Fluor647 antibody (410714, BioLegend) diluted 1:200. Dead cells were stained with the viability dye eFluor™780 (1:2000; 65-0865-14, ThermoFisher Scientific) for 30 min at 4° C. Cells were fixed with 4% paraformaldehyde before being analyzed. Flp-In™-CHO™ cells used as unstained control and single stain controls were used to determine the cut-off point between background fluorescence and positive populations. UltraComp eBeads™ Compensation Beads were used (ThermoFisher Scientific) to generate single stain controls of both anti-FLAG-iFluor647 antibody and anti-human IgG Fc-Alexa Fluor647 antibody. The data was acquired with an Attune Nxt flow cytometer (Invitrogen) and analyzed by FCS Express 7 Research Edition.

### Surface Plasmon Resonance

Surface Plasmon Resonance (SPR) was used to measure the interaction between VHHs and human or cynomolgus TfR receptor. Human TfR (2474-TR, R&D Systems) and cynomolgus TfR (90253-C07H, Sino Biological) were biotinylated with the EZ-Link NHS-PEG4-Biotinylation Kit (ThermoFischer Scientific) according to the manufacturer instructions. The binding experiments were performed at 25° C. on a Biacore T200 instrument (Cytiva, Uppsala, Sweden) in HBS-EP+ buffer (10 mM HEPES, 150 mM NaCl, 3 mM EDTA and 0.05% v/v Surfactant P20). Biotinylated human TfR and cynomolgus TfR were captured on a SA sensor chip (Cytiva) at a density of around 250 RU. Increasing concentrations of VHHs were sequentially injected in one single cycle at a flow rate of 30 μl/min. The dissociation was monitored for 20 min. No specific regeneration was needed. A reference flow was used as a control for non-specific binding and refractive index changes. Several buffer blanks were used for double referencing. Binding affinities (KD) and kinetic rate constants (kₒₙ, koff) were derived after fitting the experimental data to the 1:1 binding model with the Biacore T200 Evaluation Software 3.1 using the single cycle kinetic procedure. Each interaction was repeated a least three times.

### Bio-Layer Interferometry

Binding of the bispecific antibodies to BACE1 was assessed with an Octet RED96 (Forté Bio/Molecular Devices). Briefly, streptavidin (SA) biosensors (18-5020, Forte Bio/Molecular Devices) were pre-wet for at least 10 minutes in kinetic buffer. Next, the biosensors were dipped in biotinylated BACE1 (5 μg/ml in kinetic buffer). BACE1 (Protein Service Facility, VIB) biotinylation was performed with the EZ-Link NHS-PEG4-Biotinylation Kit (ThermoFischer Scientific) according to the manufacturer instructions. Biosensors were then sequentially submerged in baseline wells with kinetic buffer, bispecific antibodies diluted in kinetic buffer, and finally back into baseline wells to assess dissociation. Data was recorded using the Forte Bio Octet RED analysis software (Forte Bio/Molecular Devices), and sensorgrams were generated using Graphpad.

### Bispecific Engineering and Expression

1A11WT-2×Nb62 and 1A11WT were custom made by GenScript Biotech (FIG. 1A), with 1A11 being our in-house developed anti-BACE1 antibody (Zhou et al 2011 J Biol Chem 286, 8677-8687). Briefly, both the heavy chains (mouse IgG2a) and light chains (mouse kappa) was cloned into the mammalian expression vector pcDNA3.4, expressed from Expi293F™ human cells (A14527, ThermoFischer Scientific) and purified using HiTrap® MabSelect™ columns (GE11-0034-93, Cytiva). Its purity was estimated by densitometric analysis of the Coomassie Blue-stained SDS-PAGE gel under non-reducing conditions, and resulted in 75% (1A11WT-2×Nb62). The DNA encoding for 1A11AM-Nb62 (with 1A11AM being the humanized version of 1A11WT), 1A11AM-Nb188 and 1A11AM-aGFP (with aGFP being an anti-green fluorescent protein (GFP) nanobody) was synthesized by Twist Bioscience (CA, USA) and cloned in their pTwist CMV BetaGlobin WPRE Neo vector: Nb62-Fc, Nb188-Fc and aGFP-Fc (human IgG1, L234A, L235A, P329G, T350V, T366L, K392L, T394W), 1A11AM heavy chain (human IgG1, L234A, L235A, P329G, T350V, L351Y, F405A, Y407V) and 1A11AM light chain (human kappa). 1A11AM and 1A11WT bind with similar affinity to BACE1. The antibodies were expressed in Hek293F cells using the X-tremeGENE™ HP DNA Transfection Reagent (6366546001, Merck) and purified following the protocol by Nesspor et al. (2020 Sci Rep 10, 7557). The purification protocol consisted of a protein A purification, followed by a purification over a CaptureSelect™ CH1-XL Pre-packed Column (494346205, ThermoFischer Scientific).

### Sample Collection, A6 Extraction and ELISA

Mice were euthanized with a Dolethal overdose (150-200 mg/kg) injected intraperitoneally. To harvest plasma, blood was collected with a prefilled heparin syringe via cardiac puncture. Next, blood samples were spun at 2000 g for 10 minutes and plasma was collected. Brains were harvested after transcardial perfusion with heparinized PBS.

Mouse Aβ1-40 samples from brain and plasma were prepared according to Serneels et al. (2020 Mol Neurodegener 15, 60). Briefly, a brain hemisphere per mouse was homogenized in buffer containing 20 mM Tris, 250 mM sucrose, 0.5 mM EDTA, 0.5 mM EGTA (pH 7.4 HCl) supplemented with cOmplete™ protease inhibitor cocktail (Roche) using a bead mill. Next, soluble Aβ1-40 was extracted by 0.4% diethylamine treatment for 30 minutes at 4° C., high speed centrifugation (100 000 g, 1 h, 4° C.) and neutralization with 0.5 M Tris-HCl (pH 6.8). Aβ1-40 levels were quantified by ELISA using Meso Scale Discovery (MSD) 96-well plates and antibodies provided by Janssen Pharmaceutica. MAb JRFcAβ40/28, which recognizes the C-terminus of Aβ1-40, was used as a capture antibody, whereas JRF/rAβ/2 labeled with sulfoTAG as the detection antibody.

### Protein Thermal Stability Measurements

Two techniques were used to determine the melting temperatures (Tm) of proteins.

Binding of Sypro Orange (Invitrogen; Waltham, MA, USA) to hydrophobic patches was monitored upon temperature-induced unfolding in an Uncle or QuantStudio5 qPCR (Thermo Fisher; Waltham, MA, USA) instrument. 10 μl (Uncle) or 20 μl (qPCR) of sample was tested at 0.75 mg/ml (Uncle) or 0.2 mg/ml (qPCR) and a 10× Sypro Orange concentration. A linear temperature ramp was initiated from 25 to 95° C. (Uncle) or 25 to 99° C. (qPCR) at a rate of 0.5° C./min (Uncle) or 0.05° C./min (qPCR). A pre-run incubation for 180 s (Uncle) or 15 s (qPCR) was applied and excitation occurred at 473 nm (Uncle) or 520±nm (qPCR). Area under curve of fluorescence emission spectra (Uncle) or 558±11 nm (qPCR) signals were plotted against temperature. Infliction point at which the protein is half-unfolded=Tm was derived from the local minimum/maximum of the first derivative curve.

Intrinsic tryptophan-fluorescence was monitored upon temperature-induced protein unfolding in an Uncle instrument (Unchained Labs; Pleasanton, CA, USA). Here, 10 μL of sample at 1 mg/mL was applied to the sample cuvette, and a linear temperature ramp was initiated from 25 to 95° C. at a rate of 0.5° C./min, with a pre-run incubation for 180 s. The barycentric mean (BCM) and static light scattering (SLS at 266 nm and 473 nm) signals were plotted against temperature in order to obtain Tm and aggregation onset temperatures (T_{agg}), respectively.

### Protein Surface Hydrophobicity Measurements

The relative surface hydrophobicity of proteins in their folded state (Munch and Bertolotti, 2010) was assessed as follows. Sypro Orange (Invitrogen; Waltham, MA, USA) binding to hydrophobic patches at 25° C. was measured in an Uncle instrument (Unchained Labs; Pleasanton, CA, USA). 10 μL of sample at 1 mg/ml was tested in presence of a 10× Sypro Orange concentration. Pre-run incubation of 180s was followed by a linear ramp between 25 and 26° C. at a rate of 0.1° C./min. Fluorescent signals were detected between 250 and 727 nm, measurement at 25° C. was reported.

### Aggregation Assays

Analytical size-exclusion chromatography (aSEC) was carried out by applying 20 μL of a 1 mg/mL protein sample to an Agilent SEC3 (4.6 mm×300 mm) (Mw 500-150,000) column on an Agilent HPLC system (Agilent; Santa Clara, CA, USA). Samples were run in PBS at a flow rate of 0.4 mL/min. The outlet of the column was coupled to a UV detector. Retention time, recovery and percentage pre-peak are derived from UV280 measurements.

### Chemical Stability

Sample (1 mg/mL in PBS) references were stored at −80° C., whereas temperature-stressed samples were stored at 40° C. for 4 weeks. Forced oxidized samples (1 mg/mL in PBS) were supplemented with hydrogen peroxide up to a final concentration of 10 mM, followed by incubation at 37° C. for three hours, with final buffer exchange to phosphate buffered saline (PBS) using PD MidiTrap G-25 columns (GE Healthcare; Chicago, IL, USA) according to the manufacturer's instructions, and storage at −80° C. Peptide mass analysis was done at the Research Institute for Chromatography (RIC, Kortrijk, Belgium). Samples were reduced with dithiothreitol and alkylated with iodoacetamide followed by proteolytic digestion with trypsin and LysC (overnight at 25° C.). Digested samples were analyzed on RPLC-MS using a C18 RPLC column. RPLC was performed with formic acid (FA) as additive, and with H2O and acetonitrile as mobile phases. The analyses were performed on a 1290 Infinity UHPLC system (Agilent Technologies) coupled to a 6545 Q-TOF Mass Spectrometer (Agilent Technologies) operated in MS and MS/MS mode. Data processing was performed using BioConfirm 10.0 and MassHunter 7.0 (Agilent Technologies). Measured signals were matched onto the sequences. Identification was based primarily on MS-only data. Enzyme specified was trypsin (C-terminal cleavage at lysine or arginine) or LysC (C-terminal cleavage at lysine) and 0-2 missed cleavages were allowed. N-terminal cyclization (pyroglutamate from E), D isomerization, N/Q deamidation and M/W oxidation were considered as variable modifications while cysteine carbamidomethylation (sample preparation related) was considered as fixed modification while. Peak areas from extracted ion chromatograms (EICs) were used for quantifying modifications.

SEQUENCES
(full length of BBB00533)
SEQ ID No. 2
QVQLQESGGGSVQPGGSLRLSCTASGRTFNYAMGWFRQAPGKNR
EFVATIDWKDGSSYYLDSVRGRFTIERDDAKNTVYLQMNSLKPE
DAAVYTCAVGDGDYCSTYTCAAEVEYDYWGQGTQVTVSS
(CDR1 of BBB00533)
SEQ ID No. 3
GRTFNYAMG
(CDR2 of BBB00533)
SEQ ID No. 4
TIDWKDGSSY
(CDR3 of BBB00533)
SEQ ID No. 5
GDGDYCSTYTCAAEVEYDY
(full length of BBB00515)
SEQ ID No. 6
QVQLQESGGGLVQAGGSLRLSCAASGSIFSINAMGWYRQAPGKQ
RELVAVITSGGSTIYADSVKGRFTISRDNAENTVYLQMNSLKPE
DTAVYYCNAHVGLKVPTIQELSLGFGSWGQGTQVTVSS
(CDR1 of BBB00515)
SEQ ID No. 7
GSIFSINAMG
(CDR2 of BBB00515)
SEQ ID No. 8
VITSGGSTI
(CDR3 of BBB00515)
SEQ ID No. 9
HVGLKVPTIQELSLGFGS

1. A transferrin receptor (TfR) binding agent which binds to human and non-human primate TfR,
the TfR binding agent, when coupled to a chemical entity, facilitates the uptake of the chemical entity into the central nervous system (CNS) across the blood brain barrier (BBB),
wherein the TfR binding agent comprising a CDR3 sequence consisting of an amino acid sequence with maximally two amino acids different from SEQ ID No. 5 or 9 and/or wherein the TfR binding agent comprising a CDR2 sequence consisting of an amino acid sequence with maximally two amino acids different from SEQ ID No. 4 or 8 and/or wherein the TfR binding agent comprising a CDR1 sequence consisting of an amino acid sequence maximally two amino acids different from SEQ ID No. 3 or 7.

2. The TfR binding agent of claim 1, wherein the CDR3 sequence is as depicted in SEQ ID No. 5 or 9 and/or wherein the CDR2 sequence is depicted in SEQ ID No. 4 or 8 and/or wherein the CDR1 sequence is depicted in SEQ ID No. 3 or 7.

3. The TfR binding agent of claim 1 comprising an amino acid sequence with at least 90% identity over the full length of SEQ ID No. 2 or 6.

4. The TfR binding agent of claim 3 comprising an amino acid sequence depicted in any of the sequences of SEQ ID Nos. 2, 6, 11-31, and 37-68.

5. The TfR binding agent of claim 1, wherein the chemical entity is a biological, small molecule, therapeutic agent, a radionuclide, an antisense oligonucleotide, imaging agent, or test compound.

6. The TfR binding agent of claim 1, wherein the chemical entity is neurotensin, a neurotensin analogue, or an anti-BACE1 antibody.

7. The TfR binding agent of claim 1, wherein the binding agent comprises an antibody, an antibody fragment, an immunoglobulin single variable domain, or VHH.

8. The TfR binding agent of claim 1, further comprising the chemical entity that is to be transported to the CNS and/or across the BBB.

9. The TfR binding agent of claim 8, wherein the chemical entity is a neurological disorder drug, a cancer drug, or an imaging compound.

10. The TfR binding agent of claim 8, wherein the TfR binding agent transports the chemical entity across the BBB.

11. A method of delivering a chemical entity to the central nervous system (CNS) of a subject, the method comprising:
administering to the subject a TfR binding agent of claim 8.

12. The method according to claim 11, wherein the TfR binding agent transports the chemical entity across the BBB.

13. The method according to claim 11, wherein the delivery of the chemical entity to the CNS treats a neurological disorder.

14. The method according to claim 13, wherein the neurological disorder is selected from the list consisting of Alzheimer's disease, stroke, dementia, muscular dystrophy, multiple sclerosis, amyotrophic lateral sclerosis, Charcot-Marie-Tooth disease, dystonia, Parkinson's disease, viral or microbial infections, inflammation, brain cancer, neuropathic pain, and traumatic brain injury.

15. A nucleic acid molecule encoding the TfR binding agent of claim 1.

16. The nucleic acid molecule of claim 15, wherein the nucleic acid molecule is comprised in a vector.

17. The nucleic acid molecule of claim 15, wherein the nucleic acid molecule is comprised in a host cell.


SEQUENCE LISTING (68 sequences)

SEQ ID NO: 1 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 2 - protein, 127 aa, synthetic construct
QVQLQESGGGSVQPGGSLRLSCTASGRTFNYAMGWFRQAPGKNREFVATIDWKDGSSYYLDSVRGRFTIERDDAKNTVYLQMNSLKPEDAAVYTCAVGDGDYCSTYTCAAEVEYDYWGQGTQVTVSS

SEQ ID NO: 3 - protein, 9 aa, synthetic construct
GRTFNYAMG

SEQ ID NO: 4 - protein, 10 aa, synthetic construct
TIDWKDGSSY

SEQ ID NO: 5 - protein, 19 aa, synthetic construct
GDGDYCSTYTCAAEVEYDY

SEQ ID NO: 6 - protein, 126 aa, synthetic construct
QVQLQESGGGLVQAGGSLRLSCAASGSIFSINAMGWYRQAPGKQRELVAVITSGGSTIYADSVKGRFTISRDNAENTVYLQMNSLKPEDTAVYYCNAHVGLKVPTIQELSLGFGSWGQGTQVTVSS

SEQ ID NO: 7 - protein, 10 aa, synthetic construct
GSIFSINAMG

SEQ ID NO: 8 - protein, 9 aa, synthetic construct
VITSGGSTI

SEQ ID NO: 9 - protein, 18 aa, synthetic construct
HVGLKVPTIQELSLGFGS

SEQ ID NO: 10 - protein, 760 aa, Macaca fascicularis
MMDQARSAFSNLFGGEPLSYTRFSLARQVDGDNSHVEMKLGVDEEENTDNNTKANGTKPKRCGGNICYGTIAVIIFFLIGFMIGYLGYCKGVEPKTECERLAGTESPAREEPEEDFPAAPRLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEF

SEQ ID NO: 11 - protein, 126 aa, synthetic construct
EVQLVESGGGLVQPGGSLRLSCAASGSIFSINAMGWYRQAPGKQRELVAVITSGGSTIYADSVKGRFTISRDNAENTVYLQMNSLRPEDTAVYYCNAHVGLKVPTIQELSLGFGSWGQGTLVTVSS

SEQ ID NO: 12 - protein, 126 aa, synthetic construct
EVQLVESGGGLVQPGGSLRLSCAASGSIFSINAMGWYRQAPGKQRELVAVITSGGSTIYADSVKGRFTISRDNAKNTVYLQMNSLRPEDTAVYYCNAHVGLKVPTIQELSLGFGSWGQGTLVTVSS

SEQ ID NO: 13 - protein, 126 aa, synthetic construct
DVQLVESGGGLVQPGGSLRLSCAASGSIFSINAMGWYRQAPGKQRELVAVITSGGSTIYADSVKGRFTISRDNAENTVYLQMNSLRPEDTAVYYCNAHVGLKVPTIQELSLGFGSWGQGTLVTVSS

SEQ ID NO: 14 - protein, 126 aa, synthetic construct
DVQLVESGGGLVQPGGSLRLSCAASGSIFSINAMGWYRQAPGKQRELVAVITSGGSTIYADSVKGRFTISRDNAKNTVYLQMNSLRPEDTAVYYCNAHVGLKVPTIQELSLGFGSWGQGTLVTVSS

SEQ ID NO: 15 - protein, 126 aa, synthetic construct
EVQLVESGGGLVQPGGSLRLSCAASGSIFSINAMGWYRQAPGKQRELVAVITSGGSTIYADSVKGRFTISRDQAKNTVYLQMNSLRPEDTAVYYCNAHVGLKVPTIQELSLGFGSWGQGTLVTVSS

SEQ ID NO: 16 - protein, 126 aa, synthetic construct
EVQLVESGGGLVQPGGSLRLSCAASGSIFSINAMGWYRQAPGKQRELVAVITSGGSTIYADSVKGRFTISRDEAKNTVYLQMNSLRPEDTAVYYCNAHVGLKVPTIQELSLGFGSWGQGTLVTVSS

SEQ ID NO: 17 - protein, 127 aa, synthetic construct
EVQLVESGGGLVQPGGSLRLSCTASGRTFNYAMGWFRQAPGKNREFVATIDWKDGSSYYLDSVRGRFTISRDDAKNTVYLQMNSLRPEDTAVYYCAVGDGDYCSTYTCAAEVEYDYWGQGTLVTVSS

SEQ ID NO: 18 - protein, 127 aa, synthetic construct
EVQLVESGGGLVQPGGSLRLSCTASGRTFNYAMGWFRQAPGKNREFVATIDWKDGSSYYLDSVKGRFTISRDDAKNTVYLQMNSLRPEDTAVYYCAVGDGDYCSTYTCAAEVEYDYWGQGTLVTVSS

SEQ ID NO: 19 - protein, 127 aa, synthetic construct
EVQLVESGGGLVQPGGSLRLSCTASGRTFNYAMGWFRQAPGKNREFVATIDWKDGSSYYLDSVRGRFTISRDNAKNTVYLQMNSLRPEDTAVYYCAVGDGDYCSTYTCAAEVEYDYWGQGTLVTVSS

SEQ ID NO: 20 - protein, 127 aa, synthetic construct
DVQLVESGGGLVQPGGSLRLSCTASGRTFNYAMGWFRQAPGKNREFVATIDWKDGSSYYLDSVRGRFTISRDDAKNTVYLQMNSLRPEDTAVYYCAVGDGDYCSTYTCAAEVEYDYWGQGTLVTVSS

SEQ ID NO: 21 - protein, 127 aa, synthetic construct
EVQLVESGGGLVQPGGSLRLSCTASGRTFNYAMGWFRQAPGKNREFVATIDWKDGSSYYLDSVKGRFTISRDNAKNTVYLQMNSLRPEDTAVYYCAVGDGDYCSTYTCAAEVEYDYWGQGTLVTVSS

SEQ ID NO: 22 - protein, 127 aa, synthetic construct
EVQLVESGGGLVQPGGSLRLSCTASGRTFNYAMGWFRQAPGKNREFVATIDWKDGSSYYADSVKGRFTISRDDAKNTVYLQMNSLRPEDTAVYYCAVGQGDYCSTYTCAAEVEYDYWGQGTLVTVSS

SEQ ID NO: 23 - protein, 127 aa, synthetic construct
EVQLVESGGGLVQPGGSLRLSCTASGRTFNYAMGWFRQAPGKNREFVATIDWKDGSSYYADSVKGRFTISRDDAKNTVYLQMNSLRPEDTAVYYCAVGEGDYCSTYTCAAEVEYDYWGQGTLVTVSS

SEQ ID NO: 24 - protein, 127 aa, synthetic construct
EVQLVESGGGLVQPGGSLRLSCTASGRTFNYAMGWFRQAPGKNREFVATIDWKQGSSYYADSVKGRFTISRDDAKNTVYLQMNSLRPEDTAVYYCAVGDGDYCSTYTCAAEVEYDYWGQGTLVTVSS

SEQ ID NO: 25 - protein, 127 aa, synthetic construct
EVQLVESGGGLVQPGGSLRLSCTASGRTFNYAMGWFRQAPGKNREFVATIDWKQGSSYYADSVKGRFTISRDDAKNTVYLQMNSLRPEDTAVYYCAVGQGDYCSTYTCAAEVEYDYWGQGTLVTVSS

SEQ ID NO: 26 - protein, 127 aa, synthetic construct
EVQLVESGGGLVQPGGSLRLSCTASGRTFNYAMGWFRQAPGKNREFVATIDWKQGSSYYADSVKGRFTISRDDAKNTVYLQMNSLRPEDTAVYYCAVGEGDYCSTYTCAAEVEYDYWGQGTLVTVSS

SEQ ID NO: 27 - protein, 127 aa, synthetic construct
EVQLVESGGGLVQPGGSLRLSCTASGRTFNYAMGWFRQAPGKNREFVATIDWKDGSSYYADSVKGRFTISRDNAKNTVYLQMNSLRPEDTAVYYCAVGQGDYCSTYTCAAEVEYDYWGQGTLVTVSS

SEQ ID NO: 28 - protein, 127 aa, synthetic construct
EVQLVESGGGLVQPGGSLRLSCTASGRTFNYAMGWFRQAPGKNREFVATIDWKDGSSYYADSVKGRFTISRDNAKNTVYLQMNSLRPEDTAVYYCAVGEGDYCSTYTCAAEVEYDYWGQGTLVTVSS

SEQ ID NO: 29 - protein, 127 aa, synthetic construct
EVQLVESGGGLVQPGGSLRLSCTASGRTFNYAMGWFRQAPGKNREFVATIDWKQGSSYYADSVKGRFTISRDNAKNTVYLQMNSLRPEDTAVYYCAVGDGDYCSTYTCAAEVEYDYWGQGTLVTVSS

SEQ ID NO: 30 - protein, 127 aa, synthetic construct
EVQLVESGGGLVQPGGSLRLSCTASGRTFNYAMGWFRQAPGKNREFVATIDWKQGSSYYADSVKGRFTISRDNAKNTVYLQMNSLRPEDTAVYYCAVGQGDYCSTYTCAAEVEYDYWGQGTLVTVSS

SEQ ID NO: 31 - protein, 127 aa, synthetic construct
EVQLVESGGGLVQPGGSLRLSCTASGRTFNYAMGWFRQAPGKNREFVATIDWKQGSSYYADSVKGRFTISRDNAKNTVYLQMNSLRPEDTAVYYCAVGEGDYCSTYTCAAEVEYDYWGQGTLVTVSS

SEQ ID NO: 32 - protein, 109 aa, Homo sapiens
EVQLVESGGGLVQPGGSLRLSCAASGFTFSSYAMHWVRQAPGKGLEWVSVISSDGSSTYYADSVKGRFTISRDNSKNTLYLQMNSLRAEDTAVYYCARWGQGTLVTVSS

SEQ ID NO: 33 - protein, 111 aa, synthetic construct
EVQLVESGGGVVQPGGSLRLSCAASGTDFSINFIHWVRQAPGKGLEWVAGFTATGNTNYADSVKGRFTISRDNAKNTLYLQMNSLRPEDTAVYYCYMLDKWGQGTQVTVSS

SEQ ID NO: 34 - protein, 111 aa, synthetic construct
EVQLVESGGGVVQPGGSLRLSCAASGTDFSINFIRWVRQAPGKGLEWVAGFTATGNTNYADSVKGRFTISRDNAKNTLYLQMNSLRPEDTAVYYCYMLDKWGQGTQVTVSS

SEQ ID NO: 35 - protein, 111 aa, synthetic construct
EVQLVESGGGVVQPGGSLRLSCAASGTDFSINFMHWVRQAPGKGLEWVAGFTATGNTNYADSVKGRFTISRDNAKNTLYLQMNSLRPEDTAVYYCYMLDKWGQGTQVTVSS

SEQ ID NO: 36 - protein, 111 aa, synthetic construct
EVQLVESGGGVVQPGGSLRLSCAASGTDFSINFMSWVRQAPGKGLEWVAGFTATGNTNYADSVKGRFTISRDNAKNTLYLQMNSLRPEDTAVYYCYMLDKWGQGTQVTVSS

SEQ ID NO: 37 - protein, 126 aa, synthetic construct
EVQLVESGGGLVQAGGSLRLSCAASGSIFSINAMGWYRQAPGKQRELVAVITSGGSTIYADSVKGRFTISRDNAENTVYLQMNSLRPEDTAVYYCNAHVGHKVPTIQELSLGFGSWGQGTLVTVSS

SEQ ID NO: 38 - protein, 126 aa, synthetic construct
EVQLVESGGGLVQAGGSLRLSCAASGSIFSINAMGWYRQAPGKQRELVAVITSGGSTIYADSVKGRFTISRDNAENTVYLQMNSLRPEDTAVYYCNAHVGLKVPTIQELSLGHGSWGQGTLVTVSS

SEQ ID NO: 39 - protein, 126 aa, synthetic construct
EVQLVESGGGLVQAGGSLRLSCAASGSHFSINAMGWYRQAPGKQRELVAVITSGGSTIYADSVKGRFTISRDNAENTVYLQMNSLRPEDTAVYYCNAHVGLKVPTIQELSLGFGSWGQGTLVTVSS

SEQ ID NO: 40 - protein, 126 aa, synthetic construct
EVQLVESGGGLVQAGGSLRLSCAASGSIFHINAMGWYRQAPGKQRELVAVITSGGSTIYADSVKGRFTISRDNAENTVYLQMNSLRPEDTAVYYCNAHVGLKVPTIQELSLGFGSWGQGTLVTVSS

SEQ ID NO: 41 - protein, 126 aa, synthetic construct
EVQLVESGGGLVQAGGSLRLSCAASGSIFSINAMGWYRQAPGKQRELVAVITSGGSTIYADSVKGRFTISRDNAENTVYLQMNSLRPEDTAVYYCNAHHGLKVPTIQELSLGFGSWGQGTLVTVSS

SEQ ID NO: 42 - protein, 126 aa, synthetic construct
EVQLVESGGGLVQAGGSLRLSCAASGSIFSINAMGWYRQAPGKQRELVAVITSGGSTIYADSVKGRFTISRDNAENTVYLQMNSLRPEDTAVYYCNAHVGLKVPTIQELSHGFGSWGQGTLVTVSS

SEQ ID NO: 43 - protein, 126 aa, synthetic construct
EVQLVESGGGLVQAGGSLRLSCAASGSIFSINAMGWYRQAPGKQRELVAVITSGGSTIYADSVKGRFTISRDNAENTVYLQMNSLRPEDTAVYYCNAHVGLKVPTHQELSLGFGSWGQGTLVTVSS

SEQ ID NO: 44 - protein, 126 aa, synthetic construct
EVQLVESGGGLVQAGGSLRLSCAASGSIFSINAMGWYRQAPGKQRELVAVITSGGSTIYADSVKGRFTISRDNAENTVYLQMNSLRPEDTAVYYCNAHVGLKVPTIQELSLGFGSWGQGTLVTVSS

SEQ ID NO: 45 - protein, 126 aa, synthetic construct
EVQLVESGGGLVQAGGSLRLSCAASHSIFSINAMGWYRQAPGKQRELVAVITSGGSTIYADSVKGRFTISRDNAENTVYLQMNSLRPEDTAVYYCNAHVGLKVPTIQELSLGFGSWGQGTLVTVSS

SEQ ID NO: 46 - protein, 126 aa, synthetic construct
EVQLVESGGGLVQAGGSLRLSCAASGSIHSINAMGWYRQAPGKQRELVAVITSGGSTIYADSVKGRFTISRDNAENTVYLQMNSLRPEDTAVYYCNAHVGLKVPTIQELSLGFGSWGQGTLVTVSS

SEQ ID NO: 47 - protein, 126 aa, synthetic construct
EVQLVESGGGLVQAGGSLRLSCAASGSIFSINAMGWYRQAPGKQRELVAVITSGGSTIYADSVKGRFTISRDNAENTVYLQMNSLRPEDTAVYYCNAHVGLKVPTIQELHLGFGSWGQGTLVTVSS

SEQ ID NO: 48 - protein, 126 aa, synthetic construct
EVQLVESGGGLVQAGGSLRLSCAASGSIFSINAHGWYRQAPGKQRELVAVITSGGSTIYADSVKGRFTISRDNAENTVYLQMNSLRPEDTAVYYCNAHVGLKVPTIQELSLGFGSWGQGTLVTVSS

SEQ ID NO: 49 - protein, 126 aa, synthetic construct
EVQLVESGGGLVQAGGSLRLSCAASGSIFSINAMGWYRQAPGKQRELVAVITSGGSTIYADSVKGRFTISRDNAENTVYLQMNSLRPEDTAVYYCNAHVGLKVPTIHELSLGFGSWGQGTLVTVSS

SEQ ID NO: 50 - protein, 126 aa, synthetic construct
EVQLVESGGGLVQAGGSLRLSCAASGSIFSINAMGWYRQAPGKQRELVAVITSGGSTIYADSVKGRFTISRDNAENTVYLQMNSLRPEDTAVYYCNAHVGLKVPTIQELSLGFGHWGQGTLVTVSS

SEQ ID NO: 51 - protein, 126 aa, synthetic construct
EVQLVESGGGLVQAGGSLRLSCAASGHIFSINAMGWYRQAPGKQRELVAVITSGGSTIYADSVKGRFTISRDNAENTVYLQMNSLRPEDTAVYYCNAHVGLKVPTIQELSLGFGSWGQGTLVTVSS

SEQ ID NO: 52 - protein, 126 aa, synthetic construct
EVQLVESGGGLVQAGGSLRLSCAASGSIFSINAMGWYRQAPGKQRELVAVITSGGSTIYADSVKGRFTISRDNAENTVYLQMNSLRPEDTAVYYCNAHVGLKVPTIQELSLGFHSWGQGTLVTVSS

SEQ ID NO: 53 - protein, 127 aa, synthetic construct
EVQLVESGGGSVQPGGSLRLSCTASGRTFNYAMGWFRQAPGKNREFVATIDWKDGHSYYLDSVRGRFTIERDDAKNTVYLQMNSLRPEDAAVYTCAVGDGDYCSTYTCAAEVEYDYWGQGTLVTVSS

SEQ ID NO: 54 - protein, 127 aa, synthetic construct
EVQLVESGGGSVQPGGSLRLSCTASGRTFNYAMGWFRQAPGKNREFVATHDWKDGSSYYLDSVRGRFTIERDDAKNTVYLQMNSLRPEDAAVYTCAVGDGDYCSTYTCAAEVEYDYWGQGTLVTVSS

SEQ ID NO: 55 - protein, 127 aa, synthetic construct
EVQLVESGGGSVQPGGSLRLSCTASGRTFNYAMGWFRQAPGKNREFVATIDWKDGSSYYLDSVRGRFTIERDDAKNTVYLQMNSLRPEDAAVYTCAVGDGDYCHTYTCAAEVEYDYWGQGTLVTVSS

SEQ ID NO: 56 - protein, 127 aa, synthetic construct
EVQLVESGGGSVQPGGSLRLSCTASGRTFHYAMGWFRQAPGKNREFVATIDWKDGSSYYLDSVRGRFTIERDDAKNTVYLQMNSLRPEDAAVYTCAVGDGDYCSTYTCAAEVEYDYWGQGTLVTVSS

SEQ ID NO: 57 - protein, 127 aa, synthetic construct
EVQLVESGGGSVQPGGSLRLSCTASGRTFNYAMGWFRQAPGKNREFVATIDWKDGSSYYLDSVRGRFTIERDDAKNTVYLQMNSLRPEDAAVYTCAVGDGDYCSTYTCAAEVEYDHWGQGTLVTVSS

SEQ ID NO: 58 - protein, 127 aa, synthetic construct
EVQLVESGGGSVQPGGSLRLSCTASGRTFNYAMGWFRQAPGKNREFVATIDWKDHSSYYLDSVRGRFTIERDDAKNTVYLQMNSLRPEDAAVYTCAVGDGDYCSTYTCAAEVEYDYWGQGTLVTVSS

SEQ ID NO: 59 - protein, 127 aa, synthetic construct
EVQLVESGGGSVQPGGSLRLSCTASGRTHNYAMGWFRQAPGKNREFVATIDWKDGSSYYLDSVRGRFTIERDDAKNTVYLQMNSLRPEDAAVYTCAVGDGDYCSTYTCAAEVEYDYWGQGTLVTVSS

SEQ ID NO: 60 - protein, 127 aa, synthetic construct
EVQLVESGGGSVQPGGSLRLSCTASGRTFNYAMGWFRQAPGKNREFVATIDWKDGSSYYLDSVRGRFTIERDDAKNTVYLQMNSLRPEDAAVYTCAVGDGDYCSTYTCAAEVEYDYWGQGTLVTVSS

SEQ ID NO: 61 - protein, 127 aa, synthetic construct
EVQLVESGGGSVQPGGSLRLSCTASGRTFNYAMGWFRQAPGKNREFVATIDWKDGSSYYLDSVRGRFTIERDDAKNTVYLQMNSLRPEDAAVYTCAVGDGHYCSTYTCAAEVEYDYWGQGTLVTVSS

SEQ ID NO: 62 - protein, 127 aa, synthetic construct
EVQLVESGGGSVQPGGSLRLSCTASGRTFNHAMGWFRQAPGKNREFVATIDWKDGSSYYLDSVRGRFTIERDDAKNTVYLQMNSLRPEDAAVYTCAVGDGDYCSTYTCAAEVEYDYWGQGTLVTVSS

SEQ ID NO: 63 - protein, 127 aa, synthetic construct
EVQLVESGGGSVQPGGSLRLSCTASGRTFNYAMGWFRQAPGKNREFVATIDWKDGSSYYLDSVRGRFTIERDDAKNTVYLQMNSLRPEDAAVYTCAVGDGDYCSTYTCAAEVEYHYWGQGTLVTVSS

SEQ ID NO: 64 - protein, 127 aa, synthetic construct
EVQLVESGGGSVQPGGSLRLSCTASGRTFNYAMGWFRQAPGKNREFVATIDWKHGSSYYLDSVRGRFTIERDDAKNTVYLQMNSLRPEDAAVYTCAVGDGDYCSTYTCAAEVEYDYWGQGTLVTVSS

SEQ ID NO: 65 - protein, 127 aa, synthetic construct
EVQLVESGGGSVQPGGSLRLSCTASGRTFNYAMGWFRQAPGKNREFVATIDWKDGSSYYLDSVRGRFTIERDDAKNTVYLQMNSLRPEDAAVYTCAVGDGDYCSHYTCAAEVEYDYWGQGTLVTVSS

SEQ ID NO: 66 - protein, 127 aa, synthetic construct
EVQLVESGGGSVQPGGSLRLSCTASGHTFNYAMGWFRQAPGKNREFVATIDWKDGSSYYLDSVRGRFTIERDDAKNTVYLQMNSLRPEDAAVYTCAVGDGDYCSTYTCAAEVEYDYWGQGTLVTVSS

SEQ ID NO: 67 - protein, 127 aa, synthetic construct
EVQLVESGGGSVQPGGSLRLSCTASGRTFNYAMGWFRQAPGKNREFVATIDWHDGSSYYLDSVRGRFTIERDDAKNTVYLQMNSLRPEDAAVYTCAVGDGDYCSTYTCAAEVEYDYWGQGTLVTVSS

SEQ ID NO: 68 - protein, 127 aa, synthetic construct
EVQLVESGGGSVQPGGSLRLSCTASHRTFNYAMGWFRQAPGKNREFVATIDWKDGSSYYLDSVRGRFTIERDDAKNTVYLQMNSLRPEDAAVYTCAVGDGDYCSTYTCAAEVEYDYWGQGTLVTVSS